(19) United States
(12) Patent Application Publication
(10) Pub. No.: US 2026/0345891 A1
(43) Pub. Date: Nov. 5, 2026

(54) SYSTEMS AND DEVICES FOR TREATMENT OF LOCALIZED INFECTION IN AND AROUND ARTICULATING JOINTS AND METHODS OF USE

(71) Applicant: OSTEAL THERAPEUTICS, INC., Dallas, TX (US)

(72) Inventors: Daniel Pflaster, Dallas, TX (US); Brian C. de Beaubien, Dallas, TX (US); Robert C. Hutton, Dallas, TX (US); James Karl, Dallas, TX (US); Jude Paganelli, Dallas, TX (US); Todd Saunders, Dallas, TX (US)

(21) Appl. No.: 19/099,866

(22) PCT Filed: Feb. 21, 2024
(86) PCT No.: PCT/US2024/016687

§ 371 Date: Jan. 30, 2025

Related U.S. Application Data
(60) Provisional application No. 63/486,186, filed on Feb. 21, 2023.
(60) Provisional application No. 63/493,577, filed on Mar. 31, 2023.
(60) Provisional application No. 63/519,923, filed on Aug. 16, 2023.
(60) Provisional application No. 63/596,130, filed on Nov. 3, 2023.
(60) Provisional application No. 63/613,533, filed on Dec. 21, 2023.

(51) Int. Cl. A61M 3/02; A61M 1/00; A61M 3/00

(52) U.S. Cl. CPC A61M 3/0262; A61M 1/91; A61M 3/005; A61M 3/0258; A61M 3/0279; A61M 2202/0007; A61M 2202/0241

(57) ABSTRACT

A system including a cannula having a tubular portion with a lumen extending from a proximal opening to a distal opening. A distal flange surrounds the distal opening and has an outer diameter that is greater than an outer diameter of the tubular portion near the distal end region of the cannula. An open-cell sponge is included that has a first portion shaped for implantation within a wound space and a second portion sized and shaped to extend through portion. The sponge has a tensile strength sufficient to prevent the sponge from tearing during retraction of the first portion in a proximal direction into the distal opening and through the lumen of the tubular portion. Related devices, systems, kits, and methods are also provided.

### RELATED APPLICATIONS

This application claims the benefit of priority under 35 U.S.C. § 119(e) to (1) U.S. Provisional Patent Application Ser. No. 63/486,186, filed Feb. 21, 2023; (2) U.S. Provisional Patent Application Ser. No. 63/493,577, filed Mar. 31, 2023; (3) U.S. Provisional Patent Application Ser. No. 63/519,923, filed Aug. 16, 2023; (4) U.S. Provisional Patent Application Ser. No. 63/596,130, filed Nov. 3, 2023; and (5) U.S. Provisional Patent Application Ser. No. 63/613,533 filed Dec. 21, 2023. The disclosures of the patent applications are incorporated by reference herein in their entireties.

### TECHNICAL FIELD

The present disclosure generally relates to methods, systems, and devices for irrigation of biological tissue. In particular, the description relates to methods, systems, and devices for irrigation of localized infection in and around articulating joints, including infected native joints as well as debridement, antibiotics and implant retention of an infected artificial joint.

### BACKGROUND

Joint infection, including septic arthritis and periprosthetic joint infection (PJI), is a rare but devastating pathology and can be associated with severe complications. Successful management of joint infection includes surgical intervention and systemic antibiotics in the majority of cases. An important part of the surgical procedure is reduction of the bacterial bioburden. Bioburden removal is accomplished through irrigation and debridement of infected soft tissue. An infected implant may be replaced or retained at the time of treatment depending on the clinical presentation. Replacing the implant is more invasive and increases patient morbidity compared to retaining the implant, but it improves reduction of bacterial bioburden, and outcomes compared to implant retention. In chronic cases, where the infection is well established, implants are typically replaced in a one-stage or two-stage exchange arthroplasty. In acute cases, in cases of PJI in medically compromised patients, and cases of PJI involving prostheses, which are unusually difficult to remove, the most common surgical treatments retain the implants and are known as irrigation and debridement (I&D) or debridement, antibiotics and implant retention (DAIR).

Traditional systems and devices are not optimized for controlled delivery of local antibiotics during I&D, DAIR and Double DAIR procedures partly due to a lack of medical devices to maximize efficacy of irrigation and minimize the risks associated with exposure of the joint during local antibiotic therapy.

In view of the foregoing, there is a need for improved devices, systems, and methods related to controlling introduction and removal of fluids to articulating joint space.

### SUMMARY

In an implementation, provided is a system having a cannula and an open-cell sponge. The cannula includes a tubular portion having a lumen extending from a proximal opening at a proximal end region of the cannula to a distal opening at a distal end region of the cannula, the lumen having a first cross-sectional area at the proximal opening and a second, larger cross-sectional area at the distal opening. A distal flange is on the distal end region of the cannula surrounding the distal opening and having an outer diameter that is greater than an outer diameter of the tubular portion near the distal end region of the cannula. The open-cell sponge has a first portion shaped for implantation within a wound space and located distal to the distal opening of the tubular portion; and a second portion sized and shaped to extend through each of the distal opening, the lumen, and the proximal opening of the tubular portion. The sponge has a tensile strength sufficient to prevent the open-cell sponge from tearing during retraction of the first portion in a proximal direction into the distal opening and through the lumen of the tubular portion.

Upon implantation of the system in a patient, the first portion of the sponge can be located within the wound space and the second portion can be located within the lumen of the cannula such that a proximal end region of the second portion is located at an extradermal location. The distal flange can be flexible and configured to be compressed to a reduced outer diameter for removal from the wound space through an opening in a skin of the patient after removal of the sponge from the cannula. A proximal flange can be on the proximal end region of the cannula surrounding the proximal opening and have an outer diameter that is greater than an outer diameter of the tubular portion near the proximal end region of the cannula.

The system can further include a connection line configured for bidirectional or unidirectional fluid flow relative to the wound space. The system can further include a treatment delivery system fluidly coupled to the connection line and configured to control fluid flow relative to the wound space. The treatment delivery system can provide instillation and/or aspiration of the wound space. The treatment delivery system is manually or electronically controlled. The treatment delivery system can include a fluid delivery system and a control unit. The fluid delivery system can include a vacuum source. The cannula and open-cell sponge can provide fluid communication between the vacuum source and the wound space for fluid removal.

Instillation of the wound space can include delivery of a therapeutic fluid to the wound space. The therapeutic fluid can include at least one therapeutic agent. The at least one therapeutic agent can be an antibiotic, an analgesic, an antifungal, or a combination thereof. The wound space can be a joint space.

In an interrelated aspect, provided is a treatment support system including a fluid delivery system for directing a fluid to and/or from a treatment site, a portion of the fluid delivery system being configured to prevent migration of the fluid delivery system from the treatment site, the portion of the fluid delivery system being configured to collapse to a minimum cross-section for a minimally-invasive transcutaneous deployment to the treatment site or minimally-invasive transcutaneous removal from the treatment site.

The treatment support system can further include an inflatable structure configured to fill an opening of the treatment site during a treatment. The inflatable structure can include a balloon or a bladder, inflatable with gas and/or liquid. The fluid delivery system can include a catheter or tubing. The catheter or tubing can include an exterior attachment feature to removably attach the catheter or tubing to a portion of the treatment site during treatment. The exterior attachment feature can be a magnet, a suture, an adhesive, a resorbable material, a hook, and/or a ring. The treatment support system can further include a cannula that is flared or funnel shaped at a distal end at the treatment site. The cannula can include a flared internal channel and the cross-sectional area of the flared internal channel is greater at the distal end than at a proximal end of the cannula. The cannula can be a flexible biocompatible polymer.

The portion of the fluid delivery system being configured to prevent migration can be a porous component formed of a compressible open-cell foam. The porous component can have a distal end, a proximal end, and a center section, wherein each of the distal end, the proximal end, and the center section has a cross-sectional area. The porous component can be positioned with at least a portion of the center section compressed within the internal channel of the cannula and at least the distal end positioned in the treatment site. The cross-sectional area of the distal end and the cross-sectional area of the proximal end can be greater than the cross-sectional area of the portion of the center section that is compressed within the internal channel of the cannula. The porous component can be removable from the treatment site and the cannula by withdrawing the proximal component proximally through the cannula. The cross-sectional area of the distal end can be compressed by the cannula as the porous component is withdrawn. The cannula can be flexible so that the cross-sectional area of the internal channel of the cannula is reduced by compression, folding or other manipulation to facilitate removal of the distal end of the cannula from the treatment site. The system can further include an insertion tool that includes a plurality of sequentially sized slidable segments. The fluid delivery system can be a biocompatible material.

In an interrelated implementation, provided is a treatment support system including a fluid delivery system for directing a fluid to and/or from a treatment site, a portion of the fluid delivery system being configured to mechanically stabilize the treatment site while directing the fluid during articulation of the treatment site, the portion of the fluid delivery system being configured to expand from a first collapsed configuration to a second expanded stabilizing configuration during treatment and back to a collapsed configuration for removal from the treatment site. The portion of the fluid delivery system being configured to expand can be an open-cell sponge, a mesh, and/or a lattice. The mesh can include one or more flexible biocompatible materials such as a polyester, a polypropylene, a silicone, a silk, a nylon and/or a nickel-titanium alloy. The mesh can include a helically wound braided tube. The mesh can include a sleeve shaped to conform to an anatomy of a portion of the treatment site. The system can further include a spacer configured to articulate with an inner surface of the treatment site. Expansion from the first collapsed configuration to the second expanded stabilizing configuration can occur when the fluid delivery system reaches a temperature at or near patient body temperature.

In an interrelated implementation, provided is a treatment support system having a fluid delivery system for directing a fluid to and/or from a treatment site comprising a portion of a joint; and an attachment feature attached to the fluid delivery system. The attachment feature is configured to prevent migration, kinking or clogging of the fluid delivery system. The fluid delivery system further can include a catheter or tubing. The system can further include mesh having a sleeve shaped to conform and direct flow to or from an anatomy of a portion of the treatment site. The attachment feature can include a spacer configured to allow fluid flow and to protect retained implants during therapy. The spacer can be configured to mechanically stabilize the treatment site while delivering fluid to and from the treatment site during articulation of the treatment site.

In an interrelated implementation, provided is a treatment support system including a fluid delivery system for directing a fluid to and/or from a treatment site; and an irrigation device for use at the treatment site. The irrigation device has a flexible cannula having a lumen having a proximal opening and a distal opening; and a porous component including an open-cell foam having a proximal end portion and a distal end portion. The irrigation device has an assembled configuration in which the distal end portion of the porous component is positioned distal to the distal opening of the lumen of the flexible cannula and the proximal end portion of the porous component extends through the lumen such that at least a portion of the proximal end portion lies proximal to the proximal opening. The distal end portion of the porous component is sized to be retracted through the lumen of the flexible cannula.

The flexible cannula can be a biocompatible elastomer, such as silicone. The flexible cannula can be collapsible. The irrigation device can be configured to be implanted at the treatment site in the assembled configuration. The flexible cannula can be removable from the treatment site after removal of the porous component from the flexible cannula. The flexible cannula can facilitate removal of the porous component from the treatment site. The system can further include an external vacuum source. The flexible cannula and the porous component can provide fluid communication between the external vacuum source and the treatment site for fluid removal from the treatment site. The foam can have a high tensile strength to prevent tearing of the porous component during its removal through the cannula. The distal end portion of the porous component in the assembled configuration can have a cross-sectional area that is larger than a cross-sectional area of the distal opening. The distal end portion can be compressible to a smaller cross-sectional area that is smaller than the cross-sectional area of the distal opening of the flexible cannula.

In an interrelated implementation, provided is a kit for treating a localized infection in a human patient including at least one therapeutic that is an antibiotic; and an irrigation device configured to locally irrigate a treatment site of localized infection with at least one dose of the at least one therapeutic. The irrigation device includes a porous component configured to be positioned through an opening in a skin of the patient so that at least part of the porous component is positioned within the infected site and at least part of the porous component is positioned external to the skin. The porous component includes an open-cell foam or sponge. The irrigation device also includes at least one connection line for delivery of therapeutic fluid to and removal of fluid from the infected site through the porous component The kit further includes instructions for administering the at least one dose of the at least one therapeutic to treat the localized infection and for removing in a minimally-invasive manner the porous component from the treatment site through the opening in the skin after the administering. The porous component can have a tensile strength sufficient to prevent tearing during removal of the porous component from the treatment site through a minimally-invasive opening after treating. The antibiotic can include aminoglycosides, glycopeptides, cyclic lipopeptides, amikacin, cefazolin, cefepime, ampicillin, ciprofloxacin, azithromycin, doxycycline, clindamycin, vancomycin, tobramycin, gentamicin, and daptomycin. The antibiotic can be a combination of vancomycin and tobramycin. The at least one dose can be administered according to a treatment process of a control unit configured to control a fluid delivery system, the fluid delivery system configured to connect to a fluid reservoir containing the at least one dose in solution.

In an interrelated implementation, provided is a kit for managing localized pain in a human patient including at least one therapeutic that is an anesthetic or an analgesic; and an irrigation device configured to locally irrigate a treatment site of localized pain with at least one dose of the at least one therapeutic. The irrigation device includes a porous component configured to be positioned through an opening in a skin of the patient so that at least part of the porous component is positioned within the treatment site and at least part of the porous component is positioned external to the skin. The porous component includes an open-cell foam or sponge. The irrigation device includes at least one connection line for delivery of therapeutic fluid to and removal of fluid from the treatment site through the porous component. The kit includes instructions for administering the at least one dose of the at least one therapeutic to treat the localized pain and for removing in a minimally-invasive manner the porous component from the treatment site through the opening in the skin after the administering. The anesthetic can be lidocaine. The at least one therapeutic can include lidocaine and at least one antimicrobial agent. The at least one dose can be administered according to a treatment process of a control unit configured to control a fluid delivery system, the fluid delivery system configured to connect to a fluid reservoir containing the at least one dose in solution.

In an interrelated implementation, provided is a kit for managing localized antifungal therapy in a human patient including at least one therapeutic being an antifungal agent; and an irrigation device configured to locally irrigate a treatment site of localized fungal infection with at least one dose of the at least one therapeutic. The irrigation device includes a porous component configured to be positioned through an opening in a skin of the patient so that at least part of the porous component is positioned within the treatment site and at least part of the porous component is positioned external to the skin. The porous component includes an open-cell foam or sponge. The irrigation device includes at least one connection line for delivery of therapeutic fluid to and removal of fluid from the treatment site through the porous component. The kit further includes instructions for administering the at least one dose of the at least one therapeutic to treat the fungal infection and for removing in a minimally-invasive manner the porous component from the treatment site through the opening in the skin after the administering. The antifungal agent can be fluconazole. The at least one therapeutic can include fluconazole and at least one antimicrobial agent. The at least one dose can be administered according to a treatment process of a control unit configured to control a fluid delivery system. The fluid delivery system can be configured to connect to a fluid reservoir containing the at least one dose in solution.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow the disclosure are intended to define the scope of the protected subject matter.

### DESCRIPTION OF DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity. It is to be understood that devices described herein may include features not necessarily depicted in each figure.

FIG. 1 shows in schematic a treatment system fluidly coupled to a treatment delivery system;

FIG. 2A illustrates an example of a treatment system including a cannula for placement and removal of an irrigation device;

FIG. 2B shows the treatment system of FIG. 2A with a distal end of the cannula positioned against the proximal end of the porous component;

FIG. 3A is an exploded view of an irrigation device including a cannula for placement and/or removal of a porous component;

FIG. 3B is an assembled view of the irrigation device of FIG. 3A;

FIGS. 4A-4C are cross-sectional views of cannulae for use with a porous component;

FIG. 5A shows a medial parapatellar approach in a knee procedure;

FIG. 5B shows placement in the knee of FIG. 5A of the pre-assembled irrigation device of FIG. 3A;

FIG. 5C shows insertion of an instillation line relative to the irrigation device within the knee of FIG. 5B;

FIG. 5D shows a reduced patella on top of the instillation line and the irrigation device of FIG. 5C;

FIG. 5E shows a closed incision over the treatment system of FIG. 5D with the instillation line and vacuum line configured for fluidic connection to the treatment system;

FIG. 6A illustrates an example of a sequentially dilating cannula for placement and removal of an irrigation device;

FIG. 6B shows the treatment system of FIG. 6A with the sequentially dilating cannula removed;

FIG. 7A shows an example of an irrigation device having an external mesh;

FIG. 7B shows the irrigation device of FIG. 7A following collapse towards its narrow outer diameter;

FIG. 8 shows an example of a portion of an irrigation device, in accordance with some example implementations;

FIGS. 9A and 9B show an example of a sleeve for use with a treatment system in a hip, in an assembled view and in a separate view, respectively;

FIGS. 10A and 10B show an example of a sleeve for use with a treatment system in a knee, in an assembled view and in a separate view, respectively;

FIG. 11A shows an example of a treatment system for irrigation of an articulating joint, in an exploded view that additionally incorporates a spacer;

FIG. 11B shows the treatment system of FIG. 11A in cross-section;

FIG. 12A shows another example of a treatment system for irrigation of an articulating joint, in an exploded view that additionally incorporates a spacer;

FIG. 12B shows the treatment system of FIG. 12A in cross-section;

FIG. 13A shows another example of a treatment system for irrigation of an articulating joint that additionally incorporates a spacer for a knee and including a separate irrigation device;

FIGS. 13B-13D are different views of the treatment system of FIG. 13A;

FIGS. 14A-14D show different views of the expandable spacer of the treatment system of FIG. 13A.

When practical, similar reference numbers denote similar structures, features, or elements.

### DETAILED DESCRIPTION

The disclosed subject matter relates to a surgically or minimally-invasively deployed components that can improve fluid delivery to and removal from a treatment site including localized infections in and around articulating joints, during I&D (irrigation and debridement), DAIR (debridement, antibiotics and implant retention) and Double DAIR procedures.

DAIR procedures can be performed by a single surgical procedure (referred to herein as Single DAIR or DAIR) or as a planned two-stage procedure (Double DAIR). A DAIR therapy includes surgical debridement of the infected joint, often including removal of any modular components of the infected prosthesis (components not affixed to bone), sterilization and reimplantation (or replacement) of the modular components, and postoperative systemic antibiotic therapy. Septic arthritis, an infection of a joint that does not involve a prosthesis, is treated in a very similar manner in an I&D procedure, which includes surgical or minimally-invasive irrigation and debridement of the soft tissue and postoperative systemic antibiotic therapy.

The Double DAIR therapy includes a second radical soft tissue debridement and replacement of modular implant components five to seven days after the first debridement and modular component sterilization. Recent reports show DAIR efficacy improved using a Double DAIR therapeutic regimen. The first surgical procedure of a Double DAIR therapy includes debridement of soft tissue, removal, sterilization and reimplantation of the modular components and implantation of antibiotic-impregnated PMMA (polymethyl methacrylate) beads near the infected implant in an attempt to improve local antibiotic delivery to the infected joint. The second surgery of a Double DAIR therapy includes removal of the antibiotic impregnated beads, a second debridement and modular component exchange (replacement of modular components with new sterile components). The disclosed devices and systems can improve the efficacy of I&D, DAIR and Double DAIR by providing local irrigation of the infected joint with antimicrobial agents in solution, supplementing systemic antibiotic therapy and replacing the antibiotic beads in Double DAIR therapies with controlled antibiotic irrigation via an implanted temporary irrigation device.

With a significant incidence of systemic antibiotic toxicity reported in DAIR patients, controlled local irrigation provides improved safety by allowing local delivery of higher concentrations of antibiotics that can be easily and more quickly reduced or discontinued if high systemic antibiotic levels are encountered. For example, during a first stage of a Double DAIR treatment, the modular components can be removed and sterilized, radical debridement performed, and the temporary irrigation device implanted. The combination treatment can be performed over a seven-day period alternating tobramycin and vancomycin irrigation via the temporary irrigation device. During a second stage of the treatment, the temporary irrigation device is explanted, second radical debridement performed, and modular prosthesis components exchanged followed by systemic administration of postoperative antibiotics. The Double DAIR may be further improved by eliminating the need for a second stage (i.e., second debridement and local antibiotic delivery device removal) by designing the irrigation device to allow minimally-invasive removal without a second surgery. This improvement provides the advantage of local antibiotic delivery currently available only in Double DAIR with the advantage of a single surgical intervention provided by an I&D or a Single DAIR.

Irrigation of mammalian joints described herein provides flow to and from the infected joint, including delivery of any of a selection of therapeutic fluids, allowing fluid to dwell for a predetermined time, and subsequent removal of wound exudate and excess therapeutic fluids to flush the joint and reduce bioburden as part of antimicrobial therapy. The treatment delivery systems described herein can include tubular devices such as catheters and cannulae, porous material such as open-cell sponges and other meshes or lattices, and combinations of the two in which the tubular devices are in series with, internal to, external to, or side-by-side with the porous materials. Such irrigation devices can be used in practice as further described below and can be the only device used for local irrigation of therapeutic fluids or in conjunction with additional devices.

As an advantage of the proposed solution described herein, the treatment delivery system described herein can enable minimally-invasive procedures including I&D, DAIR and Double DAIR procedures. For example, one or more portions of the treatment delivery system are configured to assist placement and removal of the irrigation devices through a small incision in the skin. The treatment delivery systems described herein are configured to minimize trauma to the skin and underlying tissue and preferably eliminating the need for a surgical procedure to remove the irrigation device. As another advantage of the proposed solution described herein, the treatment delivery system can control fluid delivery to the treatment site, according to the anatomy of the treated area to ensure treatment success. Other advantages of the treatment delivery system are discussed with reference to the figures.

It should be appreciated that implementations of irrigation devices, spacers, and other components of the treatment systems described herein may be described in the context of a particular articulating joint or a particular procedure, that other articulating joints or procedures are considered as well. For example, where the device is described as being useful in the knee joint, the device can be configured for use in another articulating joint such as the hip, shoulder, elbow, wrist, ankle, etc. And where the device is described as being useful during a specific procedure such as DAIR or Double DAIR, the device can be configured for use in another procedure such as to treat a wound or a traumatic bone injury, with or without existing implants or hardware (e.g., fracture plate and screws), localized infections including surgical site infections, septic arthritis, breast implant infection, and fracture related infections. The devices described herein can be configured for use in any of a variety of procedures and treatment sites beyond those specifically called out herein.

The systems described herein can be used for delivering treatment to any of a variety of treatment sites, such as an infected joint space. Where treatment sites are referred to herein as a joint space or another site, it should be appreciated that the treatment site or type of biological tissue being treated by controlled fluid irrigation can vary, including periprosthetic tissue, joint space, bone segment, bone fracture, of any number of bones, including bones of the hip, knee, shoulder, wrist, ankle, etc. The treatment site can be a wound area, including traumatic wounds, infected tissue, surgical incision, surgical site, osteomyelitis, septic arthritis, breast implant infection, fracture-related infection, and/or infected joints. Examples of treatments include periprosthetic joint infection treatment protocols, such as DAIR and exchange arthroplasty. Although the application may be described in the context of a particular treatment site (e.g., infected joints) and connection with that site, it should be appreciated that other treatment sites are considered and the way in which the system connects with those various sites may vary.

Minimally-invasive as used herein refers to surgeries that are performed using small incisions and few stitches so that less damage is done to a patient than with a non-minimally-invasive or open surgery.

Where the fluid being delivered using the treatment systems described herein is described as an antibiotic, other fluids are considered as well, including any of a variety of irrigation fluids such as saline including irrigation fluids having one or more therapeutic capabilities including any of a variety of antimicrobials, including antibiotics, antivirals, antifungals, antiparasitics, and the like. Examples of antibiotics include aminoglycosides, glycopeptides, cyclic lipopeptides, amikacin, cefazolin, cefepime, ampicillin, ciprofloxacin, azithromycin, doxycycline, clindamycin, vancomycin, tobramycin, gentamicin, daptomycin, and combinations thereof. The fluid being delivered using the treatment systems described herein can include antifungals, alone or in combination with the antibiotic or combination of antibiotics. Examples of antifungals include azole derivatives (e.g., fluconazole, isavuconazole, Posaconazole), amphotericin B, echinocandins (e.g., anidulafungin, caspofungin, micafungin). The fluid being delivered using the treatment systems described herein can include pain medications, alone or in combination with the antibiotic or combination of antibiotics. Examples of pain medications include opioids, analgesics, anesthetics, and the like. Several classes of anesthetic and analgesics are appropriate for local irrigation of wounds, including: amino amides (e.g., lidocaine, bupivacaine, levobupivacaine, mepivacaine, ropivacaine, prilocaine), amino esters (e.g., benzocaine, chloroprocaine, procaine, tetracaine), NSAIDs (e.g., ketorolac, celecoxib, diclofenac, fenoprofen, indomethacin) and corticosteroids (e.g., prednisone, methylprednisolone, dexamethasone, triamcinolone, betamethasone, beclomethasone, flunisolide, fluticasone). The fluid being delivered using the treatment systems described herein can include a combination of antibiotics, for example, tobramycin sulfate and vancomycin HCl, and an anesthetic, such as lidocaine. The fluid being delivered using the treatment systems described herein can include a combination of antibiotics and an antifungal, such as fluconazole.

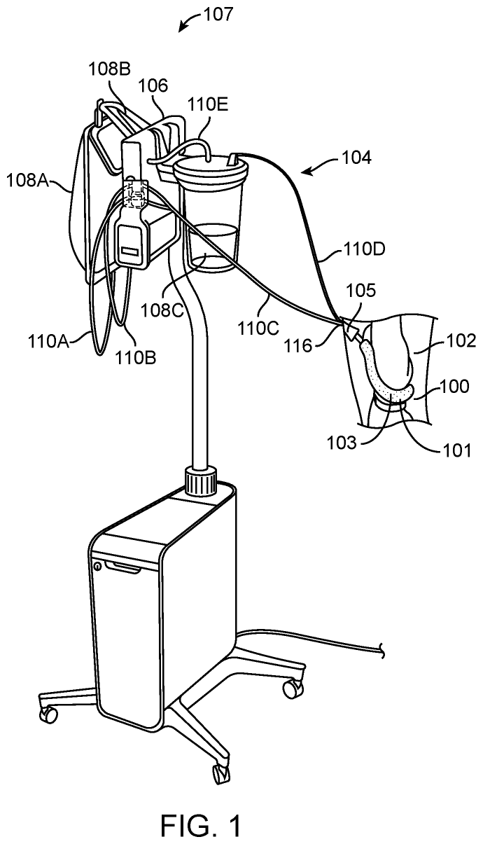

FIG. 1 shows an example of a treatment system 100 positioned at a treatment site 102 (e.g., area in and around an articulating joint). The treatment system 100 can be fluidly coupled to a treatment delivery system 107 as shown in FIG. 1. The treatment system 100 can also be coupled to a syringe or another manual component for delivery and withdrawal of fluid from the treatment site. Generally, the treatment system 100 includes an irrigation device 101 having a porous component 103 coupled to a distal end region of a connection line 105 including a catheter or tubing. The connection line 105 can be configured to bidirectional flow or unidirectional flow. The connection line 105 delivers treatment fluid from the treatment delivery system 107 to the treatment site via the porous component 103. The porous component 103 of the irrigation device 101 is configured to fill the space and/or opening of the treatment site and allow for fluid exiting the tubing to flow through the porous component 103 into the treatment site 102. The porous component 103 of the irrigation device 101 is configured to be compressed into a smaller dimension during placement and removal from the treatment site to be used in a minimally-invasive manner. Tools such as a cannula, can be insertable over the connection line 105 to minimize the cross-sectional area of the porous component 103 as the porous component 103 is advanced/withdrawn through the cannula prior to or at the completion of the irrigation treatment. The treatment system 100 can be used for a variety of methods including DAIR as described elsewhere herein or other type of healing including wound and perioperative healing.

The treatment system 100 (e.g., the connection line 105 of the irrigation device 101) can be configured to fluidly couple with the treatment delivery system 107 that can provide controlled localized delivery of fluids (e.g., antibiotics, saline, local anesthetics) into the treatment site 102 (e.g., an infected articulating joint, such as knee or hip) during, for example, I&D, DAIR, Double DAIR, or another procedure. In general, the treatment delivery system 107 can include a device for enabling adjustment of a distance between the treatment delivery system 107 and the treatment site 102 of a patient undergoing a medical treatment, such as a patient suffering from an infection treatable with fluid irrigation during I&D, DAIR and Double DAIR procedures. In the example illustrated in FIG. 1, the treatment delivery system 107 provides irrigation to the treatment site 102 including an irrigable volume (e.g., joint or bone segment) through the irrigation device 101. Although not shown, one or more other medical devices can be assisting and working in coordination and/or parallel with the treatment delivery system 107 to provide treatment for the patient (including the treatment site 102).

The treatment delivery system 107 includes a fluid delivery system 104 and a control unit 106. The fluid delivery system 104 is connected to fluid reservoir(s) 108A, 108B. The fluid delivery system 104 includes tubing, spike(s), connectors, and manual clamp(s). The fluid delivery system 104 can be loaded on the control unit 106 into normally-closed pinch valve(s), which are controlled to open and enable flow of set fluid types through the fluid lines 110A, 110B, 110C and the control unit 106. The fluid delivery system 104 includes fluid lines 110A, 110B, 110C (tubes) for directing fluid from a selected fluid reservoir 108A, 108B during a respective phase of the treatment to the treatment site and fluid lines 110D, 110E (tubes) for directing fluid from the treatment site to a collection reservoir 108C. The fluid lines 110A, 110B that transmit fluid from multiple fluid reservoir(s) 108A, 108B to the control unit 106 can be attached to the control unit 106.

The control unit 106 can control irrigation (e.g., therapeutic fluid delivery and fluid removal) of multiple fluids (e.g., irrigants) according to a set protocol including multiple phases for delivery in a particular direction of a particular fluid type for a set time duration. The settings of the control unit 106 can be predetermined for a particular treatment type (e.g., I&D, DAIR and Double DAIR procedure). The control unit 106 can enable gravity/vacuum enabled delivery of fluids (e.g., irrigants), with high precision, from fluid reservoir(s) 108A, 108B (e.g., solution bags) into the treatment site (e.g., joint space) 102 via fluid lines 110A, 110B and 110C (e.g., delivery lines). The control unit 106 can control vacuum pressure levels to remove the fluid from the treatment site 102 via fluid line 110D into the collection fluid reservoir 108C (e.g., canister).

As a route of administration, irrigation means to administer, such as by bathing or flushing, an irrigant to a treatment site, such as an open wound or a body cavity. Irrigation is bi-directional in that the irrigant that is delivered to the treatment site is also removed from the treatment site via aspiration. Delivery of a fluid to the treatment site may be referred to herein as instillation.

Some therapeutics, such as certain antibiotics, are commonly administered systemically (e.g., intravenously), which has high risk of systemic toxicity. Local instillation of an antibiotic in solution for the treatment of periprosthetic joint infection (PJI) has been reported using repeated doses of antibiotics delivered via catheter to the periprosthetic tissue or joint space following aggressive debridement and one-stage exchange of the prosthesis. Local instillation is a unidirectional delivery of the antibiotic to the treatment site in which none of the instilled antibiotic is aspirated or flushed from the treatment site. Instillation doses, therefore, are absorbed in the tissue and bloodstream such that systemic toxicity considerations also limit the local dose and concentration that may be safely administered by instillation. Whiteside et al. reported that instillation of very high concentrations of vancomycin (50,000-100,000 μg/mL) once or twice daily into the joint space resulted in serum concentrations exceeding safe limits in multiple patients, which required a reduction in the instilled dose and concentration (see Clin Orthop Relat Res (2011) 469: 26-33). Although the peak and trough concentrations of vancomycin in the joint space exceeded serum concentrations and were sustained above minimum inhibitory concentration (MIC) of vancomycin for common organisms susceptible to vancomycin, the sustained local concentration was limited by exceeding safe systemic concentrations and was not maintained at or above minimum biofilm eradication concentration (MBEC) of vancomycin (greater than 4,000 μg/mL) for multiple common PJI organisms. Similarly, the maximum daily dose provided by this instillation method was 1,000 mg.

Described herein are systems that deliver via irrigation (e.g., instillation and subsequent aspiration) one or more doses of at least one therapeutic as an irrigant (e.g., at least one therapeutic in solution) to a treatment site (e.g., periprosthetic tissue, joint space, etc.) to treat a condition (e.g., localized bacterial or fungal infection, or localized pain, etc.) at the treatment site. The systems described herein can control the treatment according to a treatment process. For example, the treatment process may locally administer in a 24-hour period a total amount of the at least one therapeutic that equals or exceeds a maximum recommended daily systemic dose for that therapeutic. The total daily dosage of the therapeutic irrigant can substantially exceed the maximum recommended daily systemic dose of the therapeutic without the toxicity risks associated with systemic administration.

In some implementations, the antibiotic vancomycin can be administered locally as an irrigant using the systems described herein at a total dose that exceeds 3,000 mg/day in a 24-hour period to treat a localized infection, for example, over 3,000 mg/day, about 4,000 mg/day, about 5,000 mg/day, about 6,000 mg/day, about 7,000 mg/day, about 7,500 mg/day up to about 40,000 mg/day. As another example, the antibiotic tobramycin can be administered locally as an irrigant using the systems at a total dose that exceeds 100 mg/day in a 24-hour period up to about 1,000 mg/day, up to about 1,250 mg/day to treat a localized infection.

As another example, the treatment process may locally administer at least one therapeutic that is an antifungal agent that is administered to locally irrigate a treatment site that is a localized fungal infection to provide the patient continuous localized antifungal therapy for at least a 24-hour period. The antifungal can be fluconazole locally administered in a 24-hour period a total amount that equals or exceeds a total daily amount permitted for systemic administration of fluconazole. The maximum recommended daily systemic dose for fluconazole is 400 mg/day. The fluconazole can be administered in combination with at least one antimicrobial agents, such as vancomycin and/or tobramycin.

As another example, the treatment process may locally administer at least one therapeutic that is an anesthetic or an analgesic that is administered to locally irrigate a treatment site to treat localized pain. The anesthetic can be lidocaine locally administered in a 24-hour period a total amount that equals or exceeds a total daily amount permitted for system administration of lidocaine. The maximum recommended daily system dose for lidocaine is 300 mg/day. The lidocaine can be administered in combination with another therapeutic as described elsewhere herein, such as with at least one antimicrobial agent.

The control unit 106 can maintain vacuum pressure levels (e.g., from approximately −5 mmHg to approximately −200 mmHg, preferably about −125 mmHg +/−10% or −112.5 mmHg to 137.5 mmHg) at particular, intermittent periods to remove the fluid from the treatment site 102 into the collection fluid reservoir 108C (e.g., canister), through a connection mechanism 116. The connection mechanism 116 can include one or more fluid connection ports to enable attachment of the fluid lines 110C, 110D for delivery to and collection of the fluids from a single irrigation device that allows bidirectional flow to/from an opening of the treatment site 102 to allow irrigation of the treatment site 102 during corresponding treatment phases. Alternatively, one or more unidirectional flow irrigation devices dedicated for inflow and one or more separate unidirectional flow irrigation devices dedicated for outflow may be used, with the inflow device(s) fluidically coupled to fluid line 110C and the outflow device(s) fluidically coupled to fluid line 110D. The control unit 106 can control fluid flow and vacuum pressure by using discrete logic components, firmware, a pressure sensor, a vacuum pump, and a solenoid pneumatic valve.

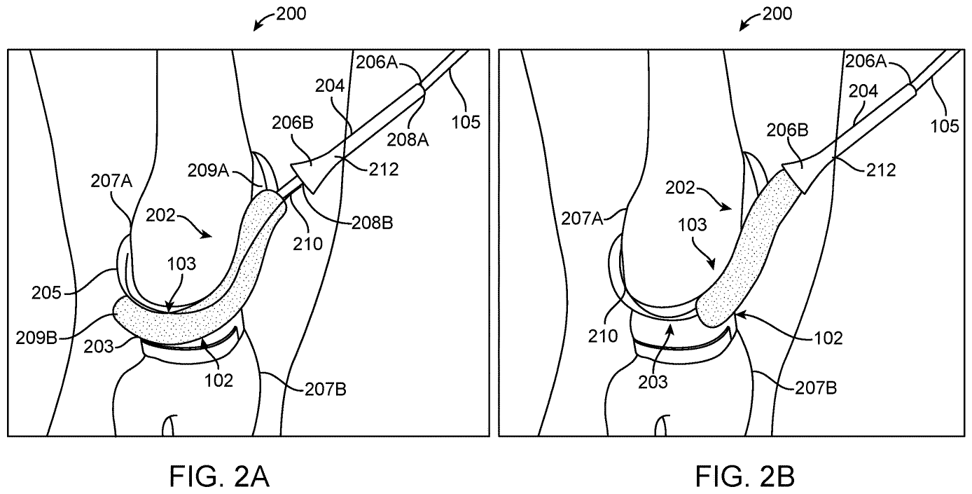

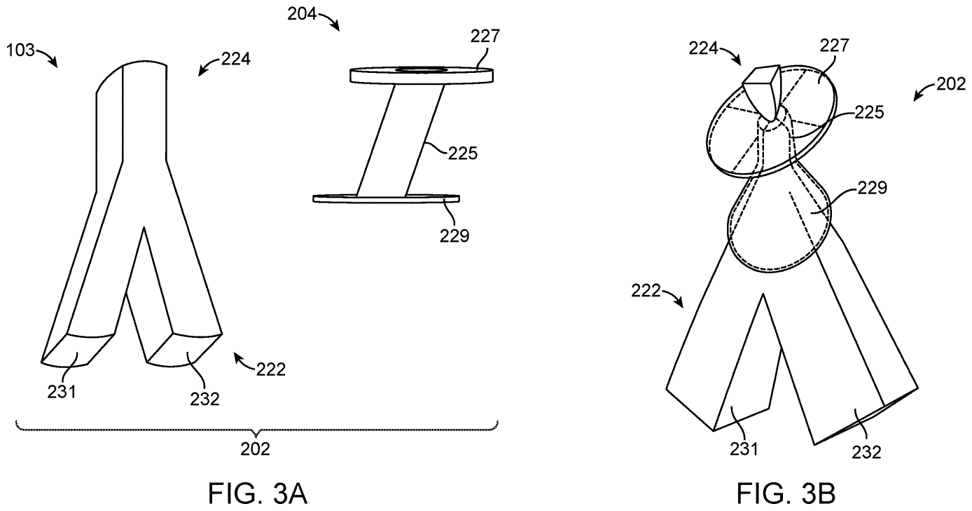

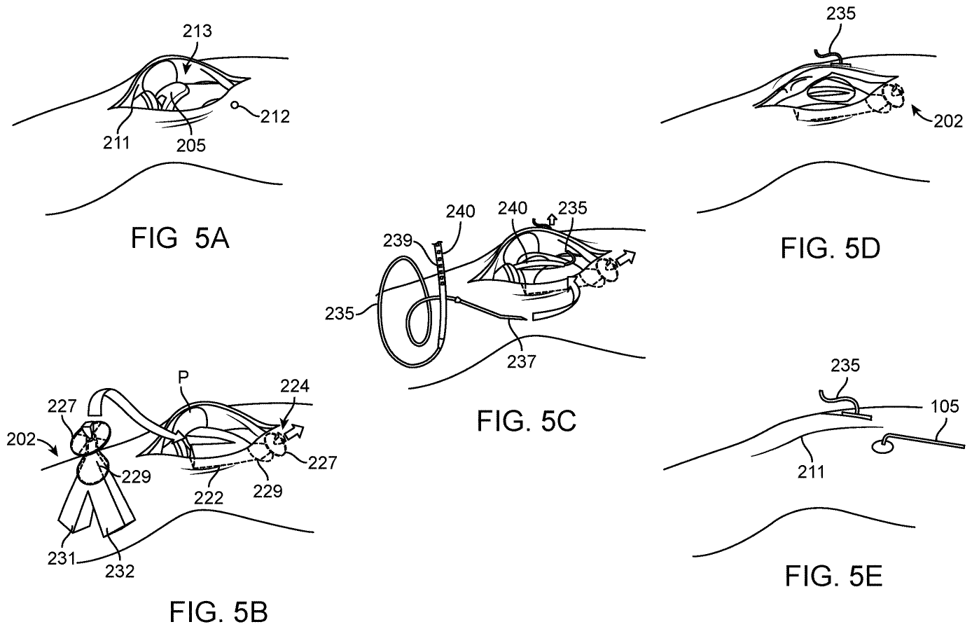

FIGS. 2A and 2B illustrate an example of a treatment system 200 including an irrigation device 202 configured to be positioned within a treatment site 102, such as an articulating joint 203 having an implant 205 in position within one or more bones 207A, 207B, and a delivery/removal cannula 204. The irrigation device 202 includes a porous component 103 and a connection line 105. The porous component 103 can be coupled to a distal end region of the connection line 105 and a flared end of the delivery cannula 204. The connection line 105 can include one or more of a catheter or tubing. The porous component 103 is shown colinear with the connection line 105. The porous component 103 in the version of FIG. 2A-2B has a tubular structure with a lumen extending through it that is sized to receive the outer diameter of the connection line 105. The porous component 103 need not have this arrangement relative to the connection line 105 and can be placed anywhere in the treatment site 102. FIGS. 3A-3B show another implementation where the porous component is not colinear with the connection line. The porous component 103 is positioned within the treatment site and the connection line is located merely adjacent the porous component 103. The connection line 105 of the implementation of FIGS. 2A-2B is bidirectional so that flow in and flow out occurs through a single lumen of a single line. The connection line 105 also can be unidirectional such that flow in and out of the site occurs through different lumens of different lines (see also FIGS. 5E, 9A-9B and FIGS. 10A-10B). In still further implementations, a connection line 105 for removal of fluid need not be incorporated at all. Rather, vacuum can be applied on a skin surface and fluid removed through an opening in the skin. The porous component 103 can be positioned so that it is fully subcutaneous and in fluid communication with the connection line 105 that is transcutaneous as shown in FIGS. 2A-2B. The porous component 103 can also be positioned so that a portion is located subcutaneously and another portion extends outside the skin for applying a vacuum on the skin surface via the connection line 105 (see FIGS. 5B-5D).

A dressing kit can be in fluid communication with the treatment site via the porous component. For example, the dressing kit can include a sterile, single use kit, including an adhesive film drape, tubing for fluid connection with a collection canister, and the porous component. Fluid can be removed from the treatment site and deposited in the canister by applying vacuum through the dressing kit. The dressing kit can include commercial dressing kits indicated for negative pressure wound therapy.

Again, with regard to FIGS. 2A-2B, the irrigation device 202 is configured to be positioned at the treatment site (e.g., knee joint, hip joint, and the like) for delivery of fluids through the connection line 105 and through the porous component 103 into the joint during a treatment regimen of the joint. The connection line 105 can include one or more distal openings (not shown) that are arranged inside or in fluid communication with the porous component 103 so that fluid delivered through the lumen of the connection line 105 is distributed through the porous component 103 and into the treatment site including the joint 203.

The porous component 103 of the irrigation device 202 can be a compressible material so that its cross-sectional area can be reduced to a smaller cross-sectional area suitable for minimally-invasive insertion of the irrigation device 202 through a small opening and expanded or allowed to return toward its original cross-sectional area once in position within the joint to fill a space for delivery a fluid treatment. Upon completion of the treatment regimen, the porous component 103 can be compressed again and removed from the joint in a minimally-invasive manner. The porous component 103 need not be inserted in a minimally-invasive manner can instead be positioned while the treatment site is open, such as described with regard to FIGS. 5A-5E.

As described above, the one or more connection lines 105 can be configured for unidirectional or bidirectional flow such that fluid is delivered to and from the joint along the same fluid path including both connection line 105 and porous component 103. In one preferred implementation, two or more connection lines 105 can be configured for unidirectional flow such that fluid is delivered to the porous component 103 and the joint through one connection line 105 and removed from the joint and porous component 103 through a different connection line 105. A connection line 105 that is unidirectional can include one line for flow into the site and another line for flow out of the site. A bidirectional connection line 105 can accommodate flow in and flow out from the treatment site. In still further implementations, no connection line 105 is used and fluid transfer out of the treatment site relies upon vacuum applied at the skin. Alternatively, any combination of connection lines 105 and porous components 103 can be configured to provide fluid flow in and out of the joint, including configurations in which only the connection line 105 is used for flow in one or both directions, and others in which only the porous component 103 is used within the joint for flow in one or both directions and is in fluid communication with the connection line 105 outside the skin.

The porous component 103 can be an open-cell foam or sponge or any type of biocompatible compressible material, including polyester, polyurethane, polyethylene, silicone, or other biocompatible materials or combinations thereof, that can be in fluid communication with the connection line 105 to enable irrigation of the joint 203. For example, the porous component 103 can include an open-cell sponge, fluid conductive channels or openings to enable irrigation of the joint 203. In some implementations, at least a portion of the connection line 105 includes uniformly-distributed openings that enable a fluid to be transmitted to the porous component 103. In some implementations, the distal end of the connection line 105, surrounded by porous component 103, includes one or more openings that enable a fluid to be transmitted to the porous component 103 to enable irrigation of the joint 203.

The porous component 103 can have an open proximal end 209A and a closed distal end 209B. The open proximal end 209A of the porous component 103 can be sized to receive a distal end of the connection line 105 (see FIGS. 2A and 2B). The closed distal end 209B of the porous component 103 can prevent the connection line 105 from extending beyond the distal end 209B of the porous component 103. The porous component 103 can have an interior dimension that can match an exterior dimension of the connection line 105 to enable a stable positioning of the porous component 103 relative to the joint 203 during the treatment.

Again, with respect to FIGS. 2A and 2B, the treatment system 200 can additionally include a cannula 204 that aids in the insertion and removal of the irrigation device 202. The cannula 204 can be placed diametrically over the connection line 105 and the porous component 103 of the irrigation device 202 positioned under the skin at the time of initial surgery to place the irrigation device 202. The cannula 204 can be introduced through a small incision 212 in the skin at the time of irrigation device removal to ease the removal of the irrigation device. The cannula 204 is preferably removed during the instillation period between insertion and removal.

The outer diameter of the cannula 204 can increase towards its distal end forming a flare or funnel-shaped feature at the distal end region 206B of the cannula 204. Similarly, the inner diameter of the cannula 204 can increase at the distal end region of the cannula 204, which may have a cylindrical shape or a flared shape outer diameter. The flare or funnel-shaped feature at the distal end region 206B gradually compresses the outer dimension of the porous structure 103 as the porous structure 103 is withdrawn into the distal end region 206B of the cannula 204 (e.g., at the time of removal of the porous structure 103 through the skin puncture 212). In some implementations, the cannula 204 can include a first, proximal end 206A with a first outer diameter that is substantially cylindrical and a second, distal end 206B with a second outer diameter, the first outer diameter being smaller than the second outer diameter. The proximal end 206A of the flared cannula can be sized to fit in a minimally-invasive opening 212 through the skin such as a surgical stab wound, approximately 6 mm in diameter. The distal end 206B of the flared cannula can be sized with a maximum inner diameter large enough to accept the cross-sectional area of a compressible porous component 103 of the irrigation device 202 (e.g., sponge or sponge covered catheter). For example, the distal end 206B of the flared cannula 204 can be approximately 10 mm or more in outer diameter, less than a maximum stretch diameter of the skin to prevent skin tear at the puncture 212. The length of the taper from the larger outer diameter to the smaller outer diameter of the flared cannula 204 can be sufficiently long to facilitate a gradual decompression of the porous component 103 from the large diameter to the small diameter as the porous component 103 is pulled through the flared cannula 204.

Still, with respect to FIGS. 2A-2B, the cannula 204 can including a proximal opening 208A and a distal opening 208B. The cannula 204 can be used to remove the irrigation device 202 from the treatment site 102. The cannula 204 can be inserted at the time of surgery such that part of the cannula 204 is positioned within the open wound of the treatment site and part of the cannula 204 is positioned outside the treatment site. The cannula 204 can be inserted by pushing the proximal end 206A through a stab incision near the open wound and urged through the stab incision until the distal end 206B is located adjacent to the porous component 103 positioned within the open wound. Because part of the surgery is open, larger components such as the flared distal end 206B can be placed in an open surgery method and the smaller components such as the proximal end 206B can be withdrawn in a minimally-invasive manner through the stab puncture. Alternatively, the flared cannula 204 can be inserted through the skin while the flared distal end 206B is constrained to a smaller outer diameter, such as within an outer sheath. The outer sheath can be withdrawn from the flared cannula 204 allowing the flared region to expand only after it is positioned subcutaneously within the treatment site.

The distal end 206B of the cannula 204 can be positioned within the treatment site 102 so that the distal opening 208B of the cannula 204 is located near or overlapping the proximal end 209A of the porous component 103 of the irrigation device 202 (see FIG. 2A). The proximal end 206A of the cannula 204 can remain outside the skin of the patient. As the The taper at the distal end 206B of the cannula 204 compresses the porous component 103 of the irrigation device 202 as it is withdrawn into the distal opening 208B and retracted through the cannula 204 towards the proximal end 206A (see FIG. 2B). The distal end 206B of the cannula 204 can taper from an outer diameter at the distal opening 208B to an outer diameter at a proximal-most end of the taper over a length of approximately 5 mm to approximately 20 mm. The distal end 206B of the cannula 204 can be flared so as to linearly taper from large to small diameter or follow a trumpet shape or an exponential shape with the greatest reduction in diameter closest to the large diameter end to rapidly compress the irrigation device 202 upon first entering the flared cannula 204.

In some implementations, the flared cannula 204, the distal end 206B of the cannula 204 or part thereof may be adjustable to facilitate placement and removal of the cannula 204 in a minimally-invasive manner, by reducing the diameter of the distal end 206B during placement or removal of the flared cannula 204 and expanding the diameter of the distal end 206B during its use to remove the irrigation device 202.

The features of the flared cannula 204 can facilitate a reduction in the cross-sectional area of the porous component 103 of the irrigation device 202 prior to removal through a minimal skin incision, according to a corresponding maximum stretched dimension, minimizing trauma to the skin and underlying tissue and preferably eliminating the need for a surgical procedure to remove the irrigation device 202. The flared cannula 204 can be constructed of one or more biocompatible materials, such as stainless steel, titanium or polymers (e.g., polyetheretherketone (PEEK), polyethylene or polyurethane) or a composite of materials in which the inner surfaces are constructed from low friction materials (e.g., Teflon or ultra-high-molecular-weight polyethylene (UHMWPE)).

The flared cannula 204 can include one or more exterior features that can be used by a surgeon to temporarily secure the flared cannula 204 in the tissue of the treatment site during DAIR treatment such that the system can remain secured in place for an extended period of time (e.g., days/weeks). The exterior features of the flared cannula 204 can include an attachment feature 210, such as a roughened surface, a porous surface, hooks, rings, flanges and other points of attachment, as well as a separate (opened or openable) ring placed over the proximal end of the irrigation device 202 to maintain the position of the irrigation device 202 at its intended location.

The porous component 103 interfaces with a flexible cannula 104 to allow fluid communication through the skin and soft tissue for fluid removal from the joint. In some implementations, the flared cannula 204 is positioned only during insertion and/or removal of the porous structure 103 and is not in position during the period between insertion and removal during fluid delivery and/or fluid removal from the treatment site. In other implementations, the flared cannula 204 can be inserted and left in place during instillation until the system is removed from the patient. The porous structure 103 and the flexible cannula 104 can remain in place within the patient during the therapy and be removed with the other components in a minimally-invasive procedure at the end of the therapy. Implementations of an irrigation device 202 and the surgical procedures for installing and removing the irrigation device 202 are described in more detail below.

FIGS. 3A-3B illustrate an implementation of an irrigation device 202 including a porous component 103 and a cannula 204, each of which are designed to be left in place during treatment. The porous component 103 is for positioning at least partially within a treatment site, such as a surgical site, a wound site, or other location. The cannula 204 is configured to maintain an opening through the skin to provide access to the porous component 103, such as for removal from the treatment site following treatment without requiring a further surgical procedure. The treatment site 102 can be a joint (e.g., knee joint, hip joint, shoulder joint, ankle joint, elbow, wrist, etc.) having an implant in position within one or more bones for delivery of fluids into and out of the joint during a treatment regimen of the joint. As discussed herein, fluid can be delivered and removed from a treatment site in a unidirectional manner through more than one lumen or connection line, or in a bidirectional manner through a single connection line. FIGS. 3A-3B illustrate an implementation in which two or more connection lines are configured for unidirectional flow such that fluid is delivered to the porous component 103 of the irrigation device 202 and the joint through a first connection line and removed from the joint and the porous component 103 through a second, different connection line The fluid delivery lines are positioned adjacent to the porous component 103.

The porous component 103 can be a compressible material, such as foam, sponge, or other type of biocompatible compressible material that can be in fluid communication with the connection line to allow for fluid transmission to or throughout the treatment site. The porous component 103 can be an open-cell sponge with fluid conductive channels or include openings that allow for fluid to be transmitted through and withdrawn from the porous component. Fluid delivered through the connection line can be distributed through the porous component 103 and into the treatment site. Fluid can also be drawn out from the treatment site through the porous component 103.

The porous component 103 is compressible such that it can be drawn through the cannula 204. Again, with respect to FIGS. 3A-3B, the porous component 103 can have a distal end region 222 and a proximal end region 224. The distal end region 222 is sized and shaped to be positioned within the treatment site. The proximal end region 224 is sized and shaped to extend through the cannula 204, which is designed to be positioned percutaneously, so that at least a portion of the proximal end region 224 of the porous component 103 is located outside the skin during use. The portion of the proximal end region 224 of the porous component 103 is positioned outside the patient while the distal end region 222 of the porous component 103 is positioned within the surgical site or wound to draw aspiration/fluid through it. The porous component 103 is preferably open-cell so that vacuum can be applied at a first region (e.g., the proximal end region 224) and be transmitted through the porous component 103 to a second region (e.g., the distal end region 222) that is spaced a distance away from the first region. The open-cells of the porous component 103 can be formed by a process of reticulation. The porous component 103 preferably has a high tensile strength so that the proximal end region 224, which is located outside the patient, can be pulled by a user to withdraw the distal end region 222 of the porous component 103, which is located inside the patient, through the cannula 204 and out of the treatment site after treatment regimen without tearing or snapping off. The tensile strength of the porous component is sufficient to prevent tearing of the porous component 103 even when one end of the porous component material is pulled and the porous component stretches and lengthens. Applying a tensile force on the material of the porous component lengthening the porous component along its long axis stretches the material, but avoids tearing the material into separate parts. The porous component 103 thus remains a unitary body during removal.

In preferred embodiments, the porous component 103 has a higher tensile strength than known polyester-based polyurethane foams, such as ROCF-V, V.A.C. VeraFlo Dressing. ROCF-V, V.A.C. VeraFlo Dressing and polyether-based polyurethanes (ROCF-G V.A.C. GranuFoam Dressing) have been tested in both wet and dry conditions (see Lessing et al., Wounds (2011); 23(10): 309-319)). ROCF-G had a tensile strength in a dry condition that was 95.44±5.17 kPa and in a wet condition that was 75.26±5.27 kPa. ROCF-V had a tensile strength in a dry condition that was 156.67±7.36 kPa and in a wet condition that was 117.48±9.08 kPa. These materials, including the ROCF-V described as having a higher tensile strength, have tensile strengths that are too low to be reliably withdrawn from the treatment site through a small opening, such as cannula 204, without tearing.

The tensile strength of the porous component material, particularly in a wet conditions, is high enough that it avoids being torn apart as one end of the material is pulled to retract the porous component 103 out from the treatment site, but not so high that the material resists compressing into a smaller outer dimension sized to slide through a minimally-invasive opening. The porous component 103 can be formed of a reticulated polyester-based polyurethane foam. The foam can have a nominal density of about 2-2.5 lbs/ft³, a pore size of about 50-60 pores per inch (ppi), and a tear strength that is greater than about 5 lbf/in, such as about 6-7 lbf/in. The tensile strength of the porous component 103 along an axis between the proximal end region and the distal end region (tested in a dry condition on a 15 mm thick sample according to ASTM D3574 Test E) is preferably greater than about 27 psi, for example, at least about 28 psi, at least about 30 psi, at least about 35 psi, at least about 40 psi, at least about 45 psi, at least about 50 psi, up to about 52 psi.

The cannula 204 can be molded from a soft and flexible material, such as biocompatible materials including silicone, polyimide, polytetrafluoroethylene (PTFE), polyurethane, polyethylene, polypropylene, polyethylene terephthalate, and nylon that is configured to conform to soft tissue at the wound site.

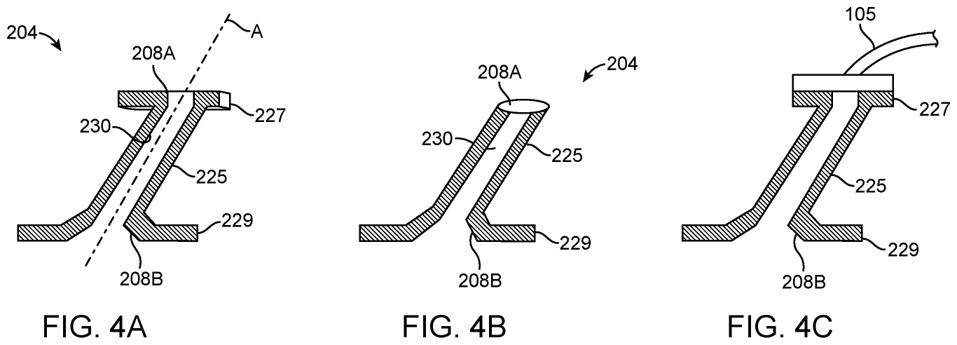

Again with respect to FIGS. 3A-3B and also FIGS. 4A-4C, the cannula 204 can be generally spool-shaped, with a single flange on one end of a cylindrical spool or two flanges, one on each end of a cylindrical spool. The cannula 204 can include a tubular central region 225 having a lumen 230. The inner lumen 230 of the central region 225 can have an inner diameter sized to allow the distal end region 222 of the porous component 103 to be pulled through the lumen 230. The outer diameter of the central cannula 225, however, is small enough to ensure the cannula 225 can span the skin without a skin puncture being stretched too large.

The central region 225 can extend between a first flange 227 on a proximal end region of the tubular element and a second flange 229 on a distal end region of the tubular element. The proximal flange 227 surrounds a proximal opening 208A into the lumen 230 of the cannula 204. The distal flange 229 surrounds a distal opening 208B into the lumen 230 of the cannula 204. The flanges 227, 229 can have any of a variety of shapes, but are generally relatively flat to conform substantially to a surface around the puncture.

As mentioned above, the cannula 204 can be positioned within the skin at the time of implanting the porous component 103 and remain in place until after the porous component 103 is removed from the patient. The flanges 227, 229 protect the skin at the wound site and the lumen 230 through the central region 225 allows the porous component 103 to be accessed from the skin exterior without surgery, even after several days of treatment for removal from the treatment site. The proximal flange 227 of the cannula 204 is arranged to sit on an external surface of the skin surrounding a skin puncture 212 (see FIG. 5B). The distal flange 229 of the cannula 204 is arranged to engage an internal surface of the soft tissue surrounding the skin puncture 212. The central region 225 has a length sufficient to substantially span the thickness of the skin and soft tissue at the location of the skin puncture 212 between the proximal flange 227 and the distal flange 229. The length of the central region 225 can be between about 1 mm and 10 mm.

The flanges 227, 229 encircling the outer diameter of the central region 225 create localized increase in an outer dimension of the cannula 204 to aid in positioning and fixing the cannula 204 during use. The distal flange 229 can be about 10 mm to about 20 mm in outer diameter and about 1 mm in thickness. The distal flange 229 is preferably flexible enough to be easily deformed for insertion through the skin and also removal through the skin without causing trauma to the puncture site 212. The proximal flange 227 can be about 10 to about 20 mm in outer diameter and about 1 mm to about 3 mm in thickness. The proximal flange 227, which is designed to remain outside of the wound site, can be more rigid than the distal flange 229. The distal flange 229, which may be referred to herein as the wound flange or the intradermal flange, can be larger in outer diameter compared to the proximal flange 227, which may be referred to herein as the skin flange. The increased outer diameter aids in preventing inadvertent withdrawal of the distal flange 229 through the skin puncture 212. The distal flange 229 can also be the same size as the outer diameter of the proximal flange 227.

The flanges 227, 229 can lie within a plane that is angled relative to the longitudinal axis A of the central region 225 (see FIGS. 4A-4C). The angle of the flanges 227, 229 relative to the central region 225 is non-perpendicular so that the lower surface of the proximal flange 227 and the upper surface of the distal flange 229 can lay substantially flush with the exterior surface and interior surface of the skin, respectively.

In some implementations, the cannula 204 has only a distal flange 229 and no proximal flange 227 (see FIG. 4B). In this implementation, the central region 225 can be longer than a thickness of the skin through which it extends. A surgeon may trim excess length on a proximal end after insertion of the cannula 204 through the skin puncture 212. Thus, a length of the central region 225 can be tailored at the time of use to the treatment site and/or patient being treated.

The proximal flange 227, if present, can be connected directly to the outflow line, such as by a removable connector (e.g., luer or Morse taper connection). The direct connection can reduce vacuum leaks and the need for occlusive dressings associated with standard wound therapy vacuum connection.

Still with respect to FIGS. 4A-4C, the length of the central region 225 between the flanges 227, 229 can vary depending on the treatment site and the size of the patient. In some implementations, the length of the central region 225 between the flanges is about 1 mm to about 3 cm. The outer diameter and/or the inner diameter of the central region 225 of the cannula 204 can change along a length of the cannula 204, for example, can be flared toward its distal end, as described elsewhere herein. The outer diameter of the central region 225 can be greater at the distal end compared to an outer diameter of the central region 225 at the proximal end. The inner diameter of the central region 225 at the distal end including the size of the distal opening 208B can be greater than the inner diameter of the central region 225 at the proximal end including the size of the proximal opening 208A.

The lumen 230 extending through the central region 225 of the cannula 204 is preferably free from any obstruction to allow for the porous component 103 to be received therein with low resistance. The inner diameter of the cannula 204 lumen 230 can be about 5 mm to about 15 mm. The inner diameter is less than the maximum cross-sectional area of the portion of the porous component 103 being withdrawn through it so that it compresses the porous component 103 moderately to reduce the size of the puncture opening, but without causing too much compression that could overcome the high tensile strength of the material of the porous component 103 and cause the porous component 103 to tear or prevent the distal end region 222 of the porous component 103 from being withdrawn through the lumen 230 during removal. For a wound site such as a knee, the cross-sectional area of the porous component 103 inside the knee joint may be larger so that the inner diameter of the lumen 230 of the cannula 204 is larger. The inner diameter of the lumen 230 of the cannula 204 is preferably sized so the outer diameter of the central region 225 is no greater than about 15 mm. The outer diameter of the cannula 204 that extends through the skin is preferably slightly larger than the incision/puncture it extends through. For example, the incision can be about 8 mm and the outer diameter of the cannula 204 where it spans the incision can be about 8.25 mm. Generally, minimally-invasive wounds in the skin are less than about 15 mm.

The lumen 230 of the cannula 204 can flare towards the distal end to better receive the larger cross-sectional area of the distal end region 222 of the porous component 103 while still helping to compress the distal end region 222 of the porous component 103 into a smaller cross-sectional area for withdrawal through the cannula 204 during removal from the patient. During removal, the porous component 103 is pulled by grasping the proximal end region 224 outside the proximal opening 208A and withdrawing the distal end region 222 through the lumen 230. Once the porous component 103 is removed through the cannula 204, the cannula 204 is then removed. The distal flange 229 of the cannula 204 can be sufficiently flexible to deform to a smaller dimension as the cannula 204 is withdrawn through the skin puncture. The presence of the proximal end region 224 of the porous component 103 within the central region 225 maintains the patency of the lumen 230 and aids in keeping the cannula 204 in place within the skin puncture 212. Once the porous component 103 is withdrawn from the cannula 204, the cannula 204 is more easily removed from the skin puncture 212.

The shape of the porous component 103 can vary depending on the anatomy being treated. Generally, at least one portion of the porous component 103 is elongated to extend outside the treatment site to an exterior of the patient. The proximal end region 224 of the porous component 103 can form an extradermal portion that is generally elongate in shape and sized to extend through the lumen 230 of the central region 225 of the cannula 204 without undergoing excessive compression by the central region 225. The distal end region 224 of the porous component 103 forming the portion positioned within the treatment site can have any of a variety of shapes depending on the demands of the anatomy in which it is to be used. As an example, during a knee replacement, the distal end region 222 of the porous component 103 that is to remain within the knee joint can incorporate at least two portions that are sized to extend over the two condyles of the knee. The two portions are preferably spaced apart from one another such that the porous component 103 does not become trapped by the patella during wound closure, providing a Y-shape to the porous component 103 as shown in FIGS. 3A-3B. The shape of the portion that is located within the treatment site can be designed to avoid the material from being trapped within the treatment space in a manner that would hinder its removal through the cannula 204. The porous component 103 of FIGS. 3A-3B includes a distal end region 222 having a first portion 231 separated a distance from a second portion 232. The first portion 231 and second portion 232 can contact the condyles of the knee on either side of the patella. The proximal end region 224 of the porous component 103 of FIGS. 3A-3B is elongate so that extends into the distal opening 208B through the lumen 230 and out the proximal opening 208A of the cannula 204.

A porous component 103 for use in a shoulder joint may have a conical, rectangular, prism shape. A porous component 103 for use in a fracture site may have a more planar shape to be placed over the bone within the wound. A porous component 103 for use in a mastectomy site, for example, may have a bulbous or generally hemispherical shape.

It should be appreciated that the cannula (e.g., cannula 204 of FIGS. 2A-2B or cannula 204 of FIGS. 3A-3B, 4A-4C) need not be fully cylindrical or have a circular cross-sectional shape. Thus, where a “diameter” of a cannula is described herein, whether it be the inner diameter or the outer diameter, it should be appreciated the cannula may have different diameters depending on where the diameter is measured. For example, the cannula can have a non-circular elongate cross-sectional shape, such as an oval or another elongate shape that has a diameter through the center along the minor axis dimension that is less than a diameter through the center along the major axis dimension. The porous structures described herein may also have a non-circular cross-sectional shape. Generally, however, the porous structure has a cross-sectional area that is larger at rest compared to the cross-sectional area when the porous structure is compressed or stretched, such as upon advancement through or pulling the porous structure from the lumen of the cannula.

FIGS. 5A-5E illustrate one method of using the irrigation device 202 of FIGS. 3A-3B. FIG. 5A illustrates an existing prosthesis 205 visible within a surgical site 213 through an open incision 211. The knee joint is being used as an example of the method, but other joints and sites of implanted devices are considered herein including hip, ankle, shoulder, elbow, wrist, spine, etc. or radius, ulna, humerus, tibia, femur, pelvis, cranium, etc. The method illustrates a medial parapatellar approach although other approaches are considered depending on the surgical site being treated. A stab incision 212 can be formed, such as with a scalpel, trocar, or other similar tool, to provide access to the supra-patellar space in the knee (FIG. 5B). The stab incision 212 can be made at an orientation such that the tool making the stab incision 212 enters the knee joint from inside the surgical site 213 distal to the open incision 211 and exits the knee joint proximal to the incision 211. A clamping tool, such as forceps, can be inserted through the stab incision 212 from an exterior surface of the skin and latched onto the proximal end region 224 of the porous component 103 of the pre-assembled irrigation device 202. The proximal end region 224 of the porous component and the proximal flange 227 of the cannula 204 can be pulled through the skin of the anterior-medial thigh (see FIG. 5B). The proximal flange 227 can lie on the skin surrounding the stab incision 212 and the distal flange 229 can lie within the wound and/or against an interior of the skin surrounding the stab incision 212. In the implementation of a knee joint, the patella P is moved away from the condyles. The proximal end region 224 of the porous component 103 extends up through the lumen 230 of the cannula 204 and the distal end region 222 of the porous component 103 lies over the knee implant 205 such that the first portion 231 and second portion 232 lie against the condyles.

The system can include separate components for delivery of fluid to the site and removal of the fluid from the site. FIG. 5C illustrates an implementation of a fluid delivery line 105 that includes a catheter or tubing 235 having a trocar tip 237 at a proximal end and a plurality of outlets 239 on a distal outflow end 240. The distal outflow end 240 having the plurality of outlets 239 allows fluid flowing through the tubing 235 from a fluid source to exit the line 105 and flow into the treatment site.

The fluid delivery line 105 is inserted by pushing the trocar tip 237 on the proximal end of the line 105 through the soft tissue, such as from inside the wound site 213 to an exterior of the skin out the anterior-medial aspect of the thigh. The trocar tip 237 facilitates surgical placement and can be sharp and configured to puncture through the skin. The tubing 235 can span the skin puncture site 212 such that the distal outflow end 240 remains within the treatment site and the proximal end is located outside the treatment site.

Once the tubing 235 is through the skin, the trocar tip 237 can be removed from the proximal end of the tubing 235. The distal outflow end 240 having the plurality of outlets 239 can be positioned over the porous component 103 (see FIG. 5C). In some implementations, the porous component 103 is Y-shaped and the distal outflow end 240 is positioned between the first and second portions 231, 232 of the distal end region 224 of the porous component 103. The fluid delivery line 105 is preferably positioned adjacent the porous component 103 to delivery fluid to the material of the component 103, but not on top of the porous component 103 to avoid trapping the porous component 103 between the anatomy and the line 105. The porous component 103 can be trimmed by a surgeon to tailor its shape relative to the anatomy and/or the fluid delivery line 105 to avoid becoming trapped within the treatment site.

The outflow end 240 can have any of a variety of cross-sectional shape including circular, oval, square, rectangular, or other shape. In some implementations, the outflow end 240 can incorporate at least one flattened surface that is designed to lie flush against the anatomy of the treatment site. The plurality of outlets 239 on the distal outflow end 240 can be located on a lower surface of the flattened surface and/or the upper surface away from the flattened surface.

Once the line 105 is properly positioned relative to the porous component 103, the patella can be reduced (see FIG. 5D) and the incision 211 closed (see FIG. 5E). The fluid delivery line 235 can be connected to a source of fluid (not shown) and a separate fluid line 105 connected to the irrigation device 202 at a distal end and a vacuum source at a proximal end. Fluid removal from the treatment site can be accomplished using vacuum pressure applied to the treatment site transmitted through the porous component 103. In some implementations, the vacuum line 105 can be coupled to the proximal flange 227 of the cannula 204 over the proximal end region 224 of the porous component 103 located external to the cannula 204 and the skin (see FIGS. 5D-5E).

After the treatment protocol period, the fluid lines 105 can be removed through the puncture. The fluid delivery line 105 can be pulled to withdraw the outflow end 240 through the skin puncture. The vacuum line 105 can be detached from the proximal flange 227 exposing the proximal end region 224 of the porous component 103. The proximal end region 224 can be grasped and pulled to withdraw the distal end region 222 of the porous component 103 located within the treatment site through the lumen 230 of the cannula 204. Once the porous component 103 is removed from the cannula 204, the flexible cannula 204 tends to collapse somewhat within the puncture 212. The cannula 204 can then be grasped and withdrawn from the puncture 212. The puncture 212 can be sized to self-seal or one or more stitches can be performed to close the puncture 212 after removal of the cannula 204.

The irrigation devices described herein, including at least the porous component, are configured to be applied to the treatment site to aid in localized therapy of the treatment site. The time-period the irrigation device is applied can vary from at least a few hours, at least 24 hours, and preferably greater than a day, at least about 2 days, at least about 3 days, at least about 4 days, at least about 5 days, at least about 6 days, at least about 7 days, or up to about 10 days before the irrigation device is removed from the treatment site. The treatment site can vary as can the treatment fluid as discussed elsewhere herein.

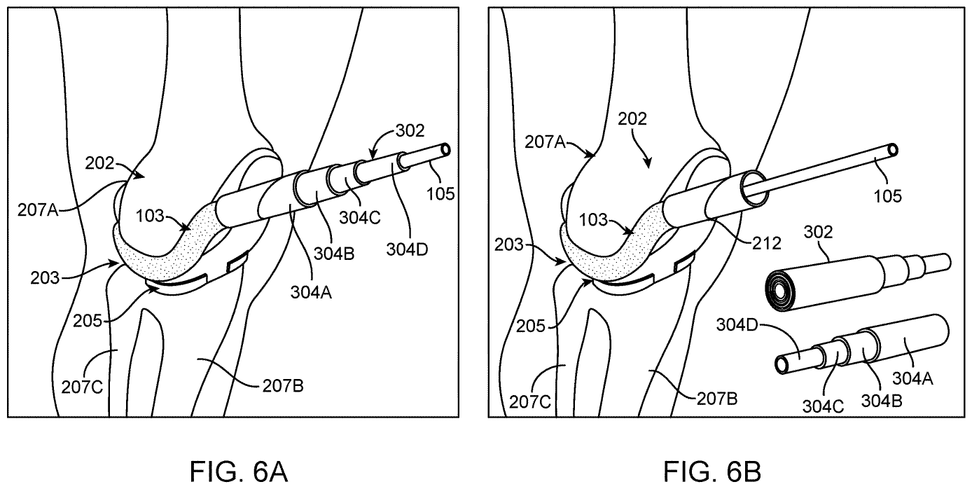

FIGS. 6A and 6B illustrate an example of a sequentially dilating set of cannulae 302 for placement and removal of an irrigation device 202, within a treatment site 102. The irrigation device 202 is shown as assembled in FIG. 6A and in an exploded view in FIG. 6B. The irrigation device 202 includes the porous component 103 and the connection line 105. The porous component 103 can be coupled to a distal end region of the connection line 105 and the sequentially dilating cannulae 302. The connection line 105 can include a catheter or tubing.

The irrigation device 202 is shown positioned so that the porous component 103 is fully within the joint under the skin incision 212 and expanded to fill the space between the femur 207A and the tibia 207B/fibula 207C. The connection line 105 extends from the porous component 103 to a region outside the skin incision 212. Cannulae 302 are shown positioned over the connection line 105 so that a distal most section of the cannulae 302 is engaged with a proximal end 209A region of the porous component 103.

The sequentially dilating cannulae 302 can be placed diametrically over the connection line 105 of the irrigation device 202 across the skin at the time the irrigation device 202 is inserted or removed to ease the transfer of the irrigation device 202. For example, each cannula of the set of cannulae 304A-D can be placed through the skin at the time of irrigation device insertion by sequentially introducing successively larger cannulae over each previously placed cannula to temporarily enlarge the wound by reversibly stretching it, without cutting or tearing the wound. The sequentially dilating cannulae 302 can facilitate removal of the irrigation device through a minimal stab wound in the skin, minimizing trauma to the skin and underlying tissue and preferably eliminating the need for a surgical procedure to remove the irrigation device 202.

The sequentially dilating cannulae 302 can include a set of cannulae 304A-D including two or more cannulae 304A-D. Alternatively, the cannulae can be a single cannula device having telescoping segments. Each cannula in the set of cannulae 304A-D can be shaped as cylinders. The cannulae in the set of cannulae 304A-D can have matching inner and outer diameters, such that the outer diameter of a first cannula matches the inner diameter of the second cannula to closely fit diametrically so that each cannula slides over the next smallest cannula with a minimal gap between them. The smallest cannula 304D of the set of cannulae 304A-D can be sized to cross, during the treatment, a minimally-invasive skin incision 212 (opening through the skin such as a surgical stab wound), of approximately 6 mm in diameter. The largest cannula 304A of the set of cannulae 304A-D can be sized with an inner diameter large enough to accept the outside diameter of an irrigation device 202. For example, the inner diameter of the largest cannula 304A in the set of cannulae 304A-D can be approximately 10 mm or more in diameter.

The sequentially dilating cannulae 302 can be constructed of one or more biocompatible materials, such as stainless steel, titanium or polymers (e.g., PEEK, Polyethylene or Polyurethane) or a composite of materials in which the inner surfaces are constructed from low friction materials (e.g., Teflon or UHMW). The sequentially dilating cannulae 302 can include one or more exterior attachment features that can be used by a surgeon to temporarily secure the sequentially dilating cannulae 302 to the joint during the treatment including extended periods of time. The exterior attachment features can include a roughened surface, a porous surface, hooks, rings, flanges and other points of attachment, as well as a separate ring placed over the proximal end of the irrigation device 202 to maintain the position of the irrigation device 202 at its intended location. The exterior attachment features can extend adjacent the porous component 103 to attach to a portion of the implant 205 or the joint 203.

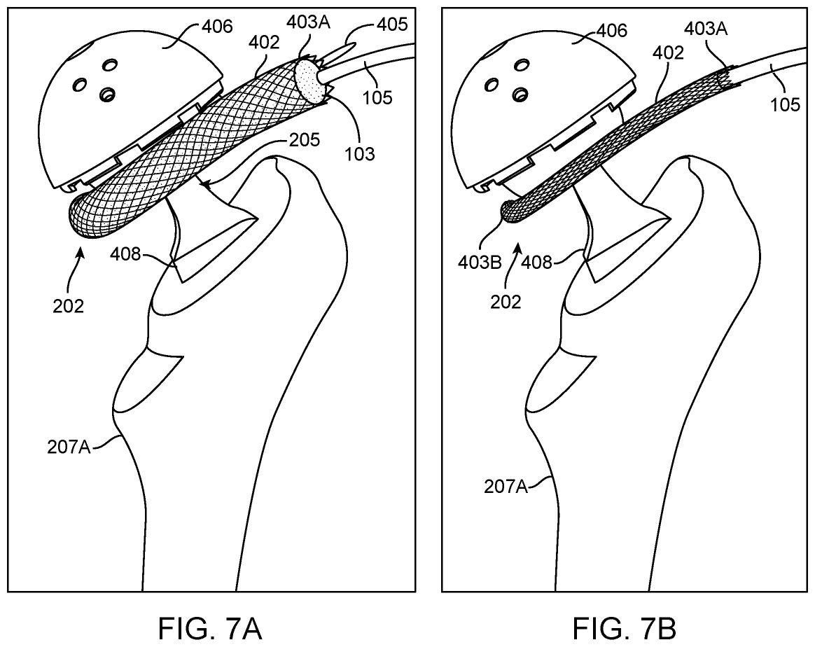

FIGS. 7A-7B show another example of an irrigation device 202 for use with the treatment system 200 including connection line 105 having a porous component 103 coupled to its distal end region and an external mesh 402, in accordance with some example implementations. The external mesh 402 can surround, as an external layer, the porous component 103 of the irrigation device 202 to control an external dimension of the porous component 103, which matches the internal dimension of the external mesh 402.

As mentioned above with regard to FIGS. 2A-2B, the porous component 103 of FIGS. 4A-4B need not be arranged coaxial with the connection line 105 as shown and can have other arrangements as described herein such that the connection line 105 may be positioned so its outlets/inlets are located near the porous component 103 to aid in the fluid delivery into and/or out from the porous component 103. Similarly, the connection line 105 can be a bi-directional connection line that is configured to deliver fluid to the treatment site and away from the treatment site. Unidirectional connection lines 105 are also considered in which one lumen or line is used to transfer fluid toward the treatment site and a second lumen or line is used to transfer fluid from the treatment site. And still further, no connection line 105 is considered such that vacuum can be applied to withdraw fluid from the treatment site through the skin.

Still with regard to FIGS. 7A-7B, external mesh 402 can be expandable so as to be expanded and collapsed at-will using a handle 405 connected to a proximal end 403A of the external mesh 402 by a link such as a mechanical linkage. The external mesh 402 is configured to be expanded during delivery of a therapeutic fluid through the irrigation device 202 into the treatment site 102 and/or aspiration of fluid from the treatment site 102 to allow the porous component 103 to be in a radially expanded state for maximized fluid transfer from connection line 105, through the porous component 103 and the external mesh 402 to the treatment site. The external mesh 402 is configured to be collapsed towards its narrow outer dimension to compress the porous component 103 within its lumen during insertion and/or removal of the irrigation device 202. The external mesh 402 can include an elongated tubular device with a plurality of openings, such as a helically wound braided tube. In an expanded form, the openings of the external mesh 402 are expanded to enable fluid delivery in a region of the treatment area 102, such as a space between the acetabular shell 406 and the stem 408 of the bone (hip) implant 205. In a collapsed form, the openings of the external mesh 402 are decreased in substantially parallel to the outer dimension of the external mesh 402 such that the external mesh 402 can be transferred through the skin incision without snagging to prevent skin tear during deployment and/or removal of the irrigation device 202. A distal end 403B of the external mesh 402 can be closed (by a continuous mesh structure) to prevent the porous component 103 from sliding outside of the external mesh 402 during insertion or removal of the irrigation device 202 or during the treatment.

The external mesh 402, the connection line 105, and the porous component 103 can vary over the length of the irrigation device 202. In some implementations, the connection line 105 (e.g., flexible catheter) can be included along the entire length of the porous component 103 and of the external mesh 402, extending from within a patient's body to outside the patient's body. In some implementations, the connection line 105 can be surrounded by the porous component 103 in one or more select areas of the irrigation device 202 corresponding to key anatomic fluid delivery or aspiration locations, further surrounded by a mesh 402 as an outermost structure.

The external mesh 402 can include slidably interwoven filaments arranged so as to reduce the overall cross-sectional area of the porous structure 103 when the proximal end of the irrigation device 202 is pulled to remove the irrigation device 202. For example, the proximal end of the connection line 105 can be pulled to start to withdraw the irrigation device 202 from the treatment site. Upon application of the pulling force in the longitudinal direction of the external mesh 402, the external mesh 402 collapses towards its narrow outer diameter and compresses the porous component 103 within its lumen. This facilitates removal of the irrigation device 202 through a minimal incision 212 in the skin, minimizing trauma to the skin and underlying tissue and preferably eliminating the need for a surgical procedure to remove the irrigation device 202. In some implementations, the connection line 105 is being withdrawn from the irrigation device 202 before the removal of the external mesh 402 and of the porous structure 103 to further decrease the outer diameter of the external mesh 402.

The external mesh 402 of the irrigation device 202 can be constructed of one or more flexible biocompatible materials such as silk, nylon or polypropylene or superelastic materials such as nickel-titanium alloys that can be coated with any biocompatible materials (e.g., Teflon or ultra-high molecular weight Polyethylene). The external mesh 402 can be a helically wound braided tube. In some implementations, the external mesh 402 can be similar to an expandable stent or a Chinese finger trap that is restricted in its ability to open, such that when tension is applied along the long axis of the braid, the braid fiber angle can be reduced, lengthening the braid while decreasing its diameter. The expansion restriction of the external mesh 402 prevents the external mesh 402 from opening before insertion or during removal. Even though the external mesh 402 of the irrigation device 202 are illustrated for irrigation around a hip, a similar external mesh 402 of the irrigation device 202 can be used for other treatment areas, such as a knee, shoulder, elbow or other joint, or in one of any variety of anatomical spaces or traumatic wounds. The irrigation device 202 can be configured to deliver unidirectional flow through one or more irrigation device 202 or bidirectional flow through a single irrigation device 202.

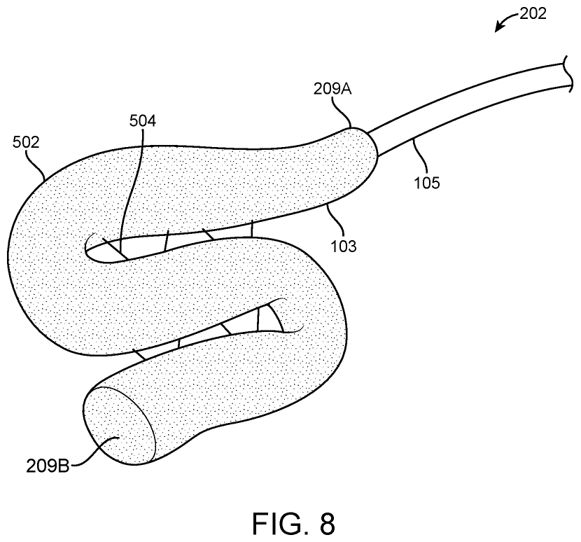

FIG. 8 shows an example of a porous structure 103 of the irrigation device 202, in accordance with some example implementations. The porous structure 103 can be configured for delivery of therapeutic fluid and/or aspiration of fluid from the body. The porous structure 103 can have an elongated tubular shape opened at the proximal end 209A to enable insertion of the connection line 105. The porous structure 103 can include an elastic sponge such as an open-cell sponge, with a particular fluid permeability to enable fluid delivery from the connection line 105 to a treatment site. The connection line 105 can include at least one structure capable of conducting fluid along the length of the irrigation device such as a tubular catheter or a cannula. The connection line 105 can be temporary or permanently attached to a lumen of the porous structure 103. For example, if the outer diameter of the connection line 105 is greater than an inner diameter of the porous structure 103, the elastic force of the expanded porous structure 103 can maintain the temporary attachment of the connection line 105 to the porous structure 103. A permanent attachment of the connection line 105 to the porous structure 103 can include a biocompatible adhesive that does not degrade or react with water or bodily fluids.

At least a portion 502 of the porous structure 103 can be shaped by bending into one or more shapes including a serpentine shape or other shape or planar surface configured to provide an increased surface area coverage and ability to fill a space of a treatment site 102 after debridement of the infected joint space. The shaped portion 502 of the porous structure 103 can be releasably secured to maintain its configuration during implantation and irrigation and be released at time of removal, so that it may be removed as a narrow structure thorough a minimally-invasive opening in the skin. The shaped portion 502 of the porous structure 103 can be secured by securing features 504 including any of magnets, sutures, hooks, adhesive, resorbable material or other means to maintain an optimal configuration of the irrigation device 202. The securing features 504 can be temporarily or permanently attached to porous structure 103. The securing features 504 that are releasably attached to porous structure 103 can be pre-manufactured for a device that is placed during an open surgical procedure, added after the porous structure 103 is inserted at the treatment site 102, or configured such that the porous structure 103 is actuated to achieve a particular shape after insertion. The securing features 504 that are attached to porous structure 103 can be sewn or otherwise attached over the entire porous structure 103 or in multiple discrete locations, as multiple sutures, adhesive bonds, etc. As shown in FIG. 8, the shaped portion 502 of the porous structure 103 is S-shaped, being maintained in a fixed configuration by the securing features 504. The securing features 504 can maintain the optimal configuration during therapy and release the shaped portion 502 of the irrigation device 202 at the completion of therapy, thereby facilitating removal of the irrigation device 202 through a minimal incision 212 in the skin, minimizing trauma to the skin and underlying tissue and preferably eliminating the need for a surgical procedure to remove the irrigation device 202. The irrigation device 202 can be used alone or in combination with the flared cannula 204 or sequential dilating cannulae 302 described with reference to FIGS. 2A-2B and 6A-6B to further facilitate removal of the irrigation device 202. The porous structure 103 need not be coaxially arranged relative to the connection line 105, as discussed elsewhere herein, and can be implanted merely adjacent the one or more connection lines 105 such as with the irrigation device 202 of FIGS. 3A-3B, 5A-5E.

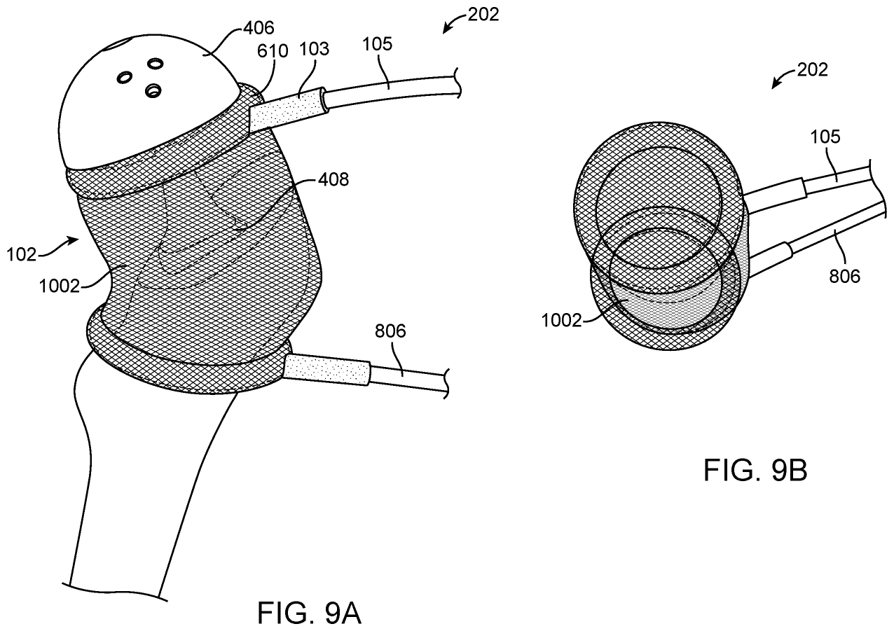

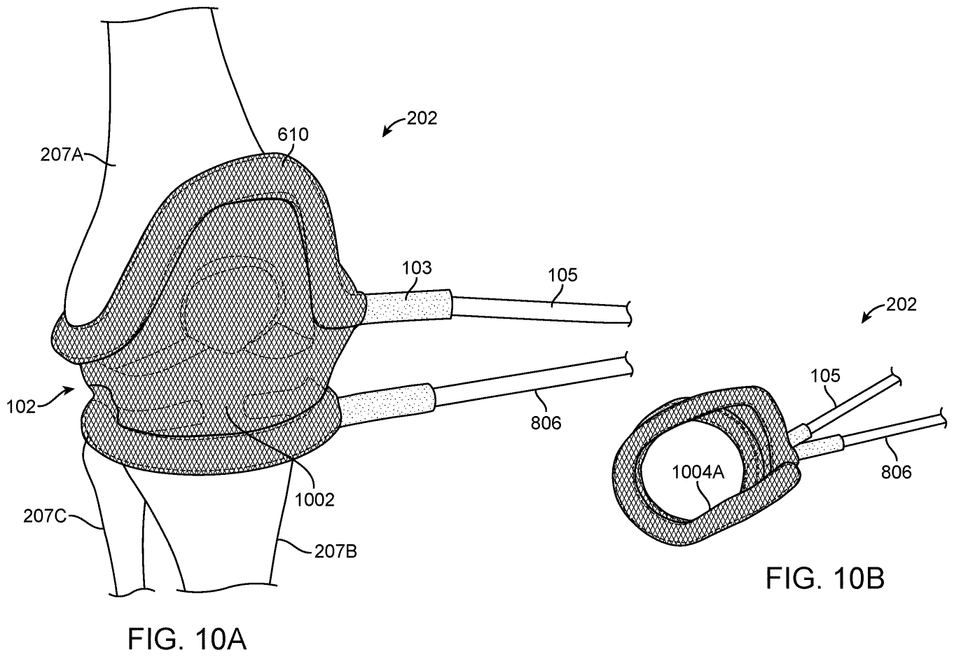

FIGS. 9A-9B and FIGS. 10A-10B illustrate a sleeve 1002 that can be used with the treatment systems described above and is configured to surround the treatment site 102. FIGS. 9A-9B illustrate the sleeve 1002 for the local irrigation of a hip and FIGS. 10A-10B illustrate the sleeve 1002 for the local irrigation of a knee.

With regard to FIGS. 9A-9B, the sleeve 1002 can include upper and lower tubular margins configured to be secured to the circular sections of the lines 105, 806. The sleeve 1002 can be expanded and collapsed at-will by adjusting a distance between the connection line 105 and the fluid line 806. Note that the connection line 105 and the fluid line 806 are unidirectional lines in which fluid is delivered to the system via one line and removed from the system via a second line. The sleeve 1002 can have a fully or partially cylindrical shape (e.g., c-shape) with upper and lower components. The sleeve 1002 is formed of a flexible material that can be collapsed or flattened during delivery to the joint space and expanded or enlarged after positioning. The upper end of the sleeve 1002 can incorporate a first component 610 configured to surround a first region of the retained implant (e.g., under the acetabular shell) and a lower end of the sleeve 1002 can incorporate a second component configured to surround a second region of the retained implant such as around an upper end of the femur as illustrated in FIGS. 9A-9B. FIGS. 10A-10B show the sleeve 1002 having an upper end incorporating a first component 610 configured to surround the lower end of the femur and a second component configured to surround a second region such as the tibial component.

The sleeve 1002 can remain extended during delivery of a therapeutic fluid through the irrigation device (e.g., via any of the irrigation devices 202 shown in FIGS. 2A-2B, 6A-6B, 7A-7B, and 8) into the treatment site 102 and/or aspiration of fluid from the treatment site 102 to secure the irrigation device 202 at the treatment site. The sleeve 1002 is configured to be detached through an opening of one of the circular sections of the lines 105, 806 and collapsed for removal from the treatment site before removal of the irrigation device 202. For example, a rip cord can be formed such as with a suture that upon pulling can result in disassembly of the mesh from the tubular portions of the device (e.g., porous component 203).

The sleeve 1002 can include a flexible substrate or mesh shaped to accommodate the joint anatomy to prevent irrigation line disruptions such as migration, kinking, and/or clogging during therapy. The flexible substrate can be woven textile constructed from a biocompatible material, such as polyester (PET), polypropylene, UHMWPE, nylon, PTFE, ePTFE, PEEK, aromatic polyester, LCP (liquid crystal polymer), elastomeric or other specialty polymers. The sleeve 1002 can be shaped to fit the treatment site (hip as illustrated in FIGS. 9A-9B or knee as illustrated in FIGS. 10A-10B) thereby reducing possibility of irrigation line disruptions during the treatment. For example, the dimensions of the sleeve 1002 can vary over the height of the irrigation device 202 to match the underlying anatomy and geometry of the treatment site 102 and the retained implant.

The sleeve 1002 can be woven with pockets 1004A to allow easy insertion and attachment foam sponge/catheter construct of the irrigation device 202. In some implementations, lines 105, 806 can be attached to the substrate of the sleeve 1002 with adhesive, sewing, radio frequency welding or other processes. The sleeve 1002 can have one or more attachment features used by a surgeon to temporarily secure the substrate of the sleeve 1002 to local soft tissue to further prevent irrigation line disruptions. The attachment features can include reinforced areas to facilitate a pass of suture for attachment to local bone or soft tissue to provide additional migration, kink, and/or clog prevention measures during treatment.

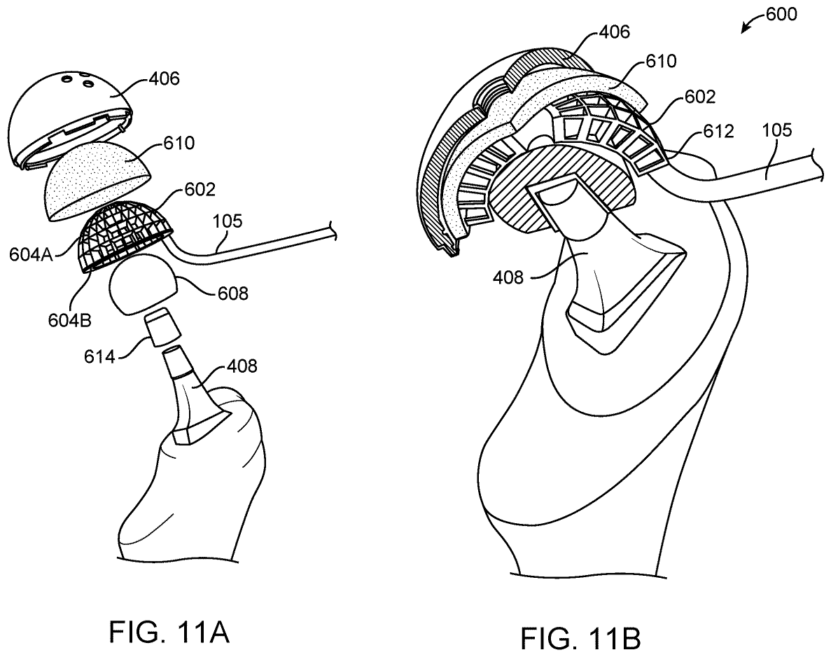

The devices and systems described above with respect to FIGS. 2A-10B are particularly useful for treating a surgical site and can be used at the time of initial surgery, for example during a DAIR procedure, that provides irrigation to the infected joint space through an expandable/compressible component that eliminates the need for a further surgical procedure to remove the irrigation device. The devices of FIGS. 11A-14D provide irrigation to the infected joint space and also occupy the space between the retained implant components to protect the surfaces during the irrigation therapy. Thus, the devices and systems of FIGS. 11A-14D are particularly useful to Double DAIR procedures or procedures in which structures are removed using another surgery.

FIGS. 11A-14D illustrate systems incorporating modular spacers that are designed to allow therapeutic fluid flow to the retained prosthesis components and the infected joint space while protecting the surfaces and locking mechanism (if present) of the retained components from mechanical damage during therapy. The spacers can be placed by a medical practitioner after removing the modular components and debridement in the first stage of a Double DAIR, thereby eliminating the need to replace or to sterilize and reinsert previously infected modular components during treatment. The spacer can be removed and replaced with definitive modular components matching the retained components during a subsequent second stage surgery of a Double DAIR.

FIGS. 11A and 11B show an example of a treatment system 600 for use in a hip where one or more modular components of the prosthesis are not retained and are temporarily replaced with system 600, in accordance with some example implementations that can be useful, for example, in a Double DAIR. A hip prosthesis can include the femoral component and the acetabular component. The femoral component is the metal ball-shaped femoral head replacing the top of the femur and is on the end of a stem or pin embedded within the femur. The acetabular component replaces the socket side of the joint and includes the metal acetabular shell and the polymer acetabular liner that articulates with the femoral head. One or more components of the hip prosthesis are fixed to the patient's bone and one or more components can be modular. For example, the femoral stem and acetabular shell are typically well-fixed to bone while the femoral head can be removed from the femoral stem and the acetabular liner can be removed from the acetabular shell.

FIG. 11A shows the treatment system 600 exploded and FIG. 11B shows the assembled system 600 in cross-section. The system 600 for the hip can include a spacer 602 and at least a first conformable porous component 610 located on an external surface of the spacer 602, a femoral head 608, and an adapter 614 (which can also be a porous component). The system 600 is sized to be received between an acetabular shell 406 of the hip prosthesis retained within the acetabulum and the stem 408 that is retained within the femur of the patient.

The spacer 602 can be hemispherical with an outer surface 604A configured to contact the porous component 610, which can be a porous component such as a foam sponge as described elsewhere herein. The spacer 602 can have an inner surface 604B configured to contact and hemispherically contain the femoral head 608 to provide stability of the joint during treatment. The spacer 602 and/or femoral head 608 can incorporate a plurality of openings (defining a cage-like structure) or channels extending between the inner and outer surfaces for therapeutic fluid flow through the spacer 602 into contact with the porous component 610 and the head 608. The spacer 602 can be in fluid communication with the retained acetabular shell 406 via the porous component 610 adjacent to the outer surface 604A of the spacer 602. The spacer 602 can be in fluid communication with the femoral head 608 adjacent to the inner surface 604B.

The spacer 602, which can be hemispherical, can be constructed out of suitable biocompatible material such as stainless steel, UHMWPE, PEEK or other low friction material. The spacer 602 can be provided in multiple outer surface diameters to allow secure fixation into the varying sizes of retained acetabular shells 406. The spacer 602 can include features such as spikes on the outer surface 604A to improve fixation to the porous component 610. Alternatively, the spacer 602 can be fixed to the porous component 610 with an adhesive. In an alternate implementation, porous component 610 and adapter 614 can be replaced with rigid solid or porous adapters specifically sized to connect the spacer 602 to retained implants of various sizes and manufacturer. The spacer 602 can include a similar function that can be constructed for the knee (not shown) to provide fluid communication with the retained components, the superior surface formed to articulate with the retained femoral component and an inferior surface designed to interface with the retained tibial component.

In the hip, as illustrated in FIGS. 11A and 11B, the porous component 610 can be placed between the modular spacer 602 and the retained acetabular shell 406 to protect the retained shell and provide a compressive fit to stabilize the modular spacer 602 in the retained acetabular shell 406 while allowing fluid communication between the modular spacer 602 and surface of the retained acetabular shell 406. The conformable nature of the porous component 610 provides a universal fit between the spacer 602 of the treatment system 600 and the variety of hip prostheses available. In some implementations, the porous component 610 can include an expandable foam to customize the porous component 610 for an improved fit. The porous component 610 can be sized and formed to fit into the retained acetabular shell 406 can be used to allow the spacer 602 to fit securely in the retained shell 406 while providing therapeutic fluid to the retained shell 406. The porous component 610 can include an open-cell foam or other biocompatible material that allows fluid flow through it while having sufficient compressive and sheer stiffness to resist loading through the hip joint during therapy. The porous component 610 can include an expanding material that can form to the retained hip or knee prosthesis in situ, further stabilizing the foam sponge in the acetabular shell 406. In some implementations, the porous component 610 can be made of shape memory material which expands to final size and shape as the temperature of the porous component 610 increases to body temperature of the patient after implantation. The biocompatible shape memory foam can be manufactured in a variety of biocompatible materials, one preferred material being open-cell polyurethane shape memory foam.

As shown in FIGS. 11A and 11B, the femoral head 608 is supported by the retained stem 408 of the hip implant 205, covered by the adapter 614. The adapter 614 can include a polymeric material, including polymeric foam and specifically shape memory foam. The adapter 614 can be locked or otherwise secured (e.g., Morse taper lock) to the stem 408. The femoral head 608 can be configured (as shape and size) to articulate with the inner surface 604B of the modular spacer 602. In some implementations, the femoral head 608 includes porous structures or channels that allow therapeutic fluid flow between the single sized spacer 602 and of the adapter 614 on the retained femoral stem 408. The adapter 614 can be constructed from a suitable material that is used to conform to and allow stable fixation of the single sized spacer 602 on multiple trunnion sizes present in commercially available femoral stems 408. In one preferred implementation, the thickness of the adapter 614, which can be a porous foam, is sufficient to allow fluid flow even when compressed between the femoral head 608 and stem 408. In another implementation, the adapter 614 can be made of shape memory foam that is preassembled to femoral head 608 and can expand to fit the trunnion of stem 408 upon reaching body temperature.

The connection line 105 can include a fluid delivery catheter. The connection line 105 can be fluidically attached to the spacer 602 to allow therapeutic fluid to be delivered through the spacer 602. For example, a distal end of the connection line 105 can be inserted in an opening 612 at a base of the spacer 602, such as the space between the inner and outer shells of the spacer 602. The opening 612 can have an inner diameter and shape matching an external diameter of the connection line 105. The opening 612 can be configured to fit a portion of the distal end of the connection line 105 with a length (e.g., 10 mm) that ensures secure connection during the treatment. The opening 612 can be in fluid connection with a center of the spacer 602, such that the fluid delivered by the connection line 105 is uniformly distributed throughout the spacer 602.

The treatment system 600 provides structural support and also ensures fluid delivery to the spaces of the joint that would otherwise be blocked by modular components of conventional implants. In some implementations, one or more separate delivery catheters or any irrigation device 202 can be used to deliver and/or aspirate therapeutic fluid. The spacer 602, which can be a separate component or integrated with femoral head 608, along with the porous component 610 and adaptor 614, allow for fluid flow through the components of the system 600 such as by having one or more regions formed of a porous material and/or by having one or more channels or interruptions from an internal surface through an external surface. The treatment system 600 can be used alone so as to connect directly to a fluid line 105 for delivery of fluid to the treatment site. The treatment system 600 also can be used in combination with any of the irrigation devices 202 described herein having a porous component 203 coupled to a connection line 205. Thus, the porous components of the treatment system 600 can further distribute fluids delivered to (and/or drawn from) the treatment site in and around the joint from the irrigation device 202. The connection line, which can be the same as used to delivery fluid to the site, can be incorporated to also aspirate fluid from the treatment site.

The treatment system 600 is described above as incorporating a fluid connection between the connection line 105 and the spacer 602. In an alternate implementation, the fluidic connection can be made between connection line 105 and the porous femoral head 608 in the same manner as described for the spacer 602 herein. And still further, the femoral head 608 and the spacer 602 can be integrated as a single component such that the connection line 105 is coupled to a region of the spacer/head component.

In an alternate implementation (not shown), spacer 602 is not included and femoral head 608 articulated directly with porous component 610. In said implementation, porous component 610 can be configured to extend further around head 608 to provide stability to the joint and fill empty space around the retained implants.

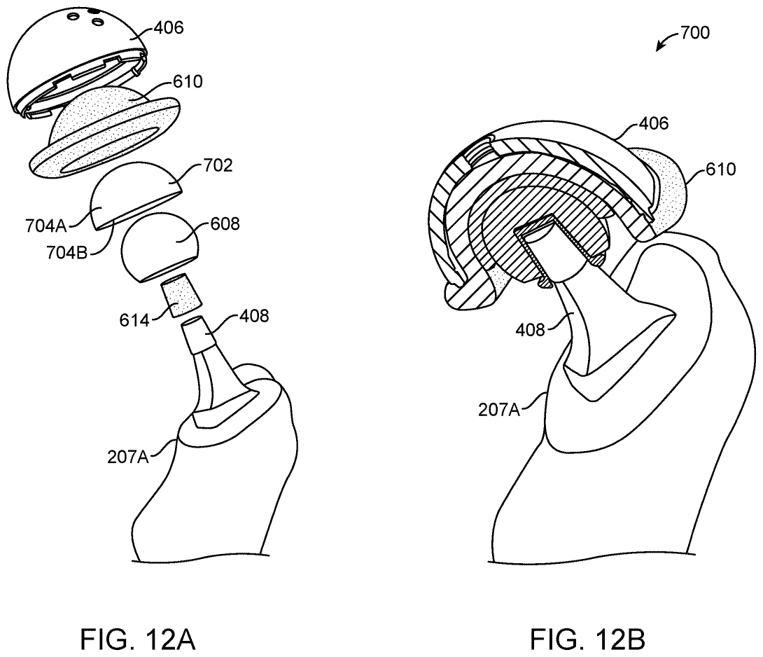

FIGS. 12A and 12B show an example of a treatment system 700 for use in a hip where one or more modular components of the prosthesis are not retained and are temporarily replaced with system 700, in accordance with some example implementations that can be useful, for example, in a Double DAIR. The system 700 for the hip can include a spacer 702, at least a first porous component 610 located on an external surface of the spacer 702, a femoral head 608 and an adapter 614. The system 700 is sized to be received between an acetabular shell 406 of the hip prosthesis retained within the acetabulum of the patient and the stem 408 retained within the femur.

The spacer 702 can be hemispherical with an outer surface 704A and an inner surface 704B. The outer surface 704A articulates with the porous component 610, which can be a shape memory sponge. As discussed above, the treatment systems can be used in combination with any of the irrigation devices 202 described herein incorporating a porous component coupled to the fluid line. In this implementation of the treatment system, the outer surface 704A can be configured to leave a lip section of the porous component 610 protruding outside the circumference of the hemispherical spacer 702 to enable fluid conduction around the irrigation device 202. The spacer 702 can have an inner surface 704B configured to contact and to articulate with the femoral head 608. The two articular outer surfaces 704A, 704B of the hemispherical spacer 702 can increase the joint range of motion and reduce a chance of dislocation. The hemispherical spacer 702 can constrain the ball head to cover a motion arc of greater than 180 degrees requiring the spacer and ball head to be pre-assembled or assembled by the surgeon in situ, further reducing a chance of hip dislocation. The hemispherical spacer 702 can be constructed out of suitable biocompatible material (e.g., stainless steel, UHMWPE, PEEK) or other low friction material. The porous component 610 can be provided in multiple outer surface diameters and include surface features such as spikes to allow secure fixation into the varying sizes of retained acetabular shells 406.

The porous component 610 can be placed between the hemispherical spacer 702 and the retained acetabular shell 406 to protect the retained shell and provide a compressive fit to stabilize the hemispherical spacer 702 in the retained acetabular shell 406 while allowing fluid communication between the joint space and surface of the retained acetabular shell 406. In some implementations, the porous component 610 can include an expandable foam to customize the foam sponge for an improved fit. The porous component 610 can be sized and formed to fit into the retained acetabular shell 406 and can be used to allow the spacer 702 to fit securely in the retained shell 406 while providing therapeutic fluid to the retained shell 406. The porous component 610 can include an open-cell foam or other biocompatible material that allows fluid flow through it while having sufficient compressive and sheer stiffness to resist loading through the hip joint during therapy. The porous component 610 can include an expanding material that can form to the retained hip or knee prosthesis in situ, further stabilizing the porous component 610 in the acetabular shell 406. In some implementations, the porous component 610 can be made of shape memory material which expands to final size and shape after being placed by the surgeon as the temperature increases to body temperature after implantation.

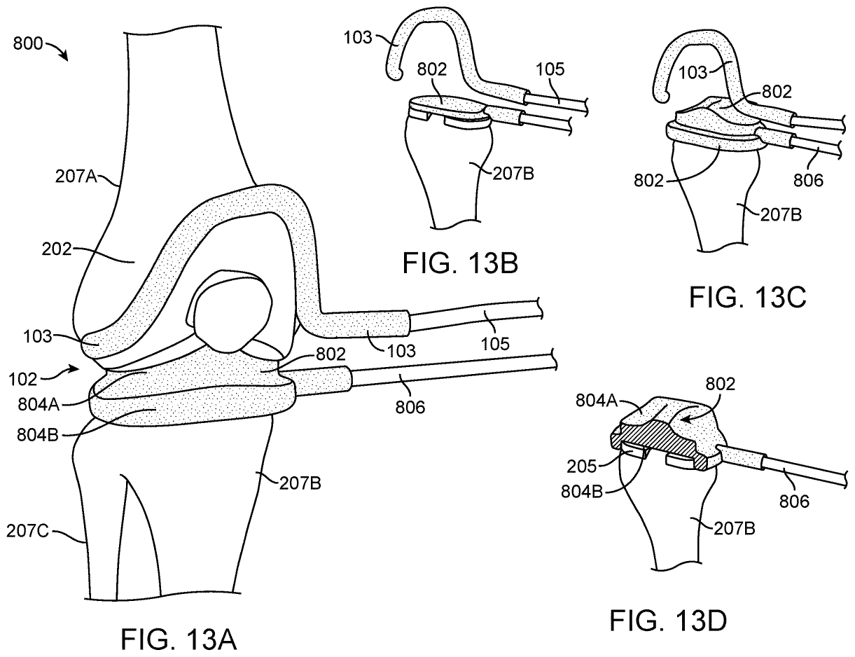

FIGS. 13A-13D shows an example of a treatment system 800 for a treatment site 102, where one or more modular components of the prosthesis are not retained and are temporarily replaced with system 800. FIG. 13A illustrates the treatment system 800 used in combination with an irrigation device 202 including a porous structure 103 coupled to a region of the connection line 105. The implant 205 is retained in position within one or more bones 207A, 207B, 207C. A flexible spacer 802 is positioned between the implant components. The flexible spacer 802 is configured to fill a space of the treatment site 102, for example, after modular component removal during a DAIR treatment. As with hip prostheses, a knee implant can also have modular components. A knee implant typically includes at a minimum a femoral component and a tibial component and a plastic spacer between them that is modular and removable. In conventional implants, the plastic spacer is not porous and thus blocks delivery of fluid to the infected implant surfaces to be treated. The treatment system 800 shown in FIGS. 13A-13D incorporate porous components that can improve delivery and/or removal of fluids from the joint space and implant surfaces.

FIG. 13B illustrates the tibial component of the implant and a spacer 802 positioned over it. The irrigation device 202 includes a porous component 103, which may have a hook shape matching the femoral portion of the implant 205, and a connection line 105 coupled to the porous component 103. The porous component 103 can be coupled to a distal end region of the connection line 105 or contain the connection line 105 within over some or all of the length of porous component 103. The irrigation device 202 is configured to be positioned around a lower portion of the femur 207A surrounding the femoral portion of the implant 205 for delivery of fluids through the connection line 105 and/or through the porous component 103 into the joint during a treatment regimen of the joint. For example, porous component 103 can include fluid conductive channels or openings to enable irrigation of the joint 203. In some implementations, at least a portion of the connection line 105 includes uniformly distributed openings that enable a fluid to be transmitted to the porous component 103 or directly to the joint space. In some implementations, an end of the connection line 105, surrounded by porous component 103, includes one or more openings that enable a fluid to be transmitted to the porous component 103 to enable irrigation of the joint 203. The interior (lumen) diameter of the porous component 103 can match an exterior diameter of the connection line 105 to enable a stable positioning of the porous component 103 relative to the joint 203 during the treatment. The porous component 103 of the irrigation device 202 can be a compressible material so that its cross-sectional area can be minimized during insertion of the irrigation device 202 through a small skin incision and expanded once in position within the treatment site 102 to fill a space for delivery a fluid treatment. Upon completion of the treatment regimen, the porous component 103 can be compressed again and removed from the joint in a minimally-invasive manner as discussed in detail above with regard to FIGS. 2A-5.

The flexible spacer 802 can include a superior surface 804A and an inferior surface 804B. The superior surface 804A can be flat when the flexible spacer 802 is deflated, as shown in FIG. 13B. The superior surface 804A can be formed to articulate with a bottom surface of the retained femoral component of the implant 205, when inflated, as shown in FIGS. 13A, 13C, and 13D. The superior surface 804A can have a wavy shape, being thicker in the middle than on the sides, when inflated. The inferior surface 804B can be flat when the flexible spacer 802 is deflated, as shown in FIG. 13B. The inferior surface 804B can be designed to interface with the retained tibial component of the implant 205 by expanding over at least a margin of the retained tibial component of the implant 205. The flexible spacer 802 can be deflated so that its outer thickness can be minimized during insertion of the spacer and expanded once in position within the treatment site 102 to fill a space for support and for delivery a fluid treatment by the irrigation device 202. Upon completion of the treatment regimen, flexible spacer 802 can be deflated again and removed from the joint in a minimally-invasive manner.

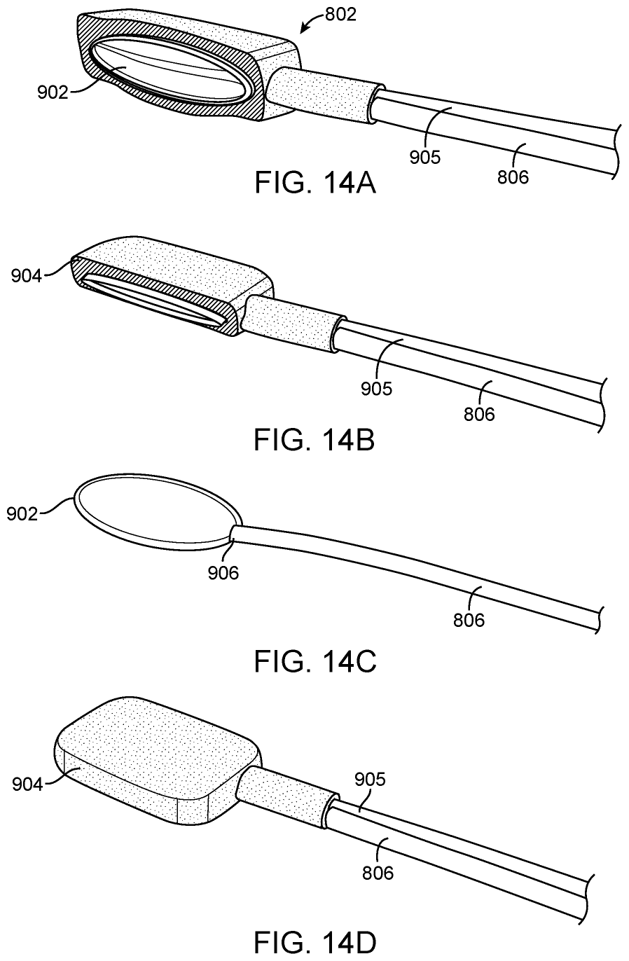

FIGS. 14A-14D show the flexible spacer 802 of FIGS. 13A-13D. The flexible spacer 802 can include an inflatable bladder 902 that is surrounded by a porous component 904, such as a foam sponge or any type of biocompatible compressible material that can expand to enable support of the retained implant 205 and stability of the joint during the treatment. The inflatable bladder 902 can be used to adjust the size of the sponge inserted between retained hip or knee implants to accommodate varying component size and anatomy. The inflatable bladder 902 can be connected to the fluid line 806 for controlling the fluid pressure within the inflatable bladder 902 and therein the volume of the inflatable bladder 902. The porous component 904 can be connected to another fluid line 905 for delivery of irrigation fluid to the treatment site.

FIG. 14A shows the inflated bladder 902 in cross section. FIG. 14B shows the bladder 902 in cross-section prior to inflation. FIG. 14C shows the bladder without the outer porous component 904. FIG. 14D shows the outer porous component 904 covering the bladder 902 and prior to inflation. The fluid used to inflate inflatable bladder 902 can include sterile saline solution or other biocompatible fluid (gas and/or liquid) that can be added or withdrawn from the bladder 902 via a sealable connection 906 between the fluid (liquid or gas) source and the bladder 902. The fluid source can be a syringe. The coverage of the porous component 904 on the expandable bladder 902 can be continuous or discontinuous. In the continuous implementation, the porous component 904 can be applied to the bladder 902 and packaged in a compressed configuration, so that when the bladder 902 can be expanded in situ. The porous component 904 can expand in conjunction with the bladder 902 at the interface between the two components. In some implementations, only a portion of the inflatable bladder 902 can be covered by the porous component 904, such that areas of the inflatable bladder 902 with porous component coverage can have a restricted expansion or can expand less than uncovered regions of the bladder 902.

In an alternate implementation of FIGS. 14A-14D, bladder 902 is not included and the function of expanding porous component 904 is accomplished by it being made of biocompatible shape memory foam which expands as the foam reaches body temperature after implantation. In another implementation, the bladder 902 can be filled with shape memory foam that expands the bladder 902 as the bladder temperature rises to body temperature. In another implementation, an additional fluid line can be fluidically coupled to the porous structure 904 for delivery of therapeutic fluid and/or aspiration of fluid from the body.

The treatment system 800 of FIGS. 12A-12D as well as the treatment system 600 of FIGS. 11A-11B and the treatment system 700 of FIGS. 12A-12B can be used with an irrigation device 202 such as the devices shown in FIGS. 2A-10B. If the irrigation device 202 is incorporated with the system to pass fluid to the joint space, a separate tube (i.e., tube 105 of FIGS. 11A-11B or tube 905 of FIGS. 14A-14D) need not be incorporated. Instead, the treatment systems can rely on fluid delivery (and/or removal) through a single tube (e.g., delivery line 105) to the irrigation device 202. Irrigation provided by the devices of FIGS. 2A-10B to the joint space may be sufficient to expose all pathogens in the joint to therapeutic fluid. However, to improve the fluid delivery/removal, the treatment system can incorporate one or more porous components to substitute for one or more of the modular components of the prosthesis that would otherwise block fluid delivery to and from the implant site. Combining the treatment system 800 having one or more porous modular components with the porous components of the irrigation device 202 provides an additional level of fluid communication to the system.

One or more conduits can be used for the delivery of treatment fluid to the treatment site and one or more conduits can be used for outflow of fluid from the treatment site. The same conduit can be used for the delivery of treatment fluid to the treatment site as is used for removal of fluid from the treatment site. Still further, no separate conduit need be incorporated for removal of fluid from the treatment site. Instead, vacuum can be performed at the skin surface to remove fluid from the treatment site.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, ”an implementation,“ or the like, means that a particular feature, structure, configuration, or characteristic described is included in at least one embodiment or implementation. Thus, the appearance of the phrase ”one embodiment,“ ”an embodiment,“ ”one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

In accordance with some implementations of the disclosed subject matter, manufactured articles, devices, systems and methods are provided for controlling fluid delivery to a treatment site during debridement, antibiotics and implant retention (DAIR) and Double DAIR procedures.

In one aspect, a treatment support system includes a fluid delivery system for directing a fluid to and from a treatment site, a portion of the fluid delivery system being configured to prevent migration of the fluid delivery system from the treatment site, the portion of the fluid delivery system being configured to collapse to a minimum cross-section for a minimally-invasive transcutaneous deployment to the treatment site or minimally-invasive transcutaneous removal from the treatment site.

In some variations, one or more features disclosed herein including the following features can optionally be included in any feasible combination. The treatment support system can further include an inflatable structure configured to fill an opening of the treatment site during a treatment. The inflatable structure can include a balloon or a bladder, inflatable with gas and/or liquid. The fluid delivery system can include a catheter or tubing. The catheter or tubing can include an exterior attachment feature to removably attach the catheter or tubing to a portion of the treatment site during treatment. The exterior attachment feature can include a magnet, a suture, an adhesive, a resorbable material, a hook, and/or a ring. The treatment support system can further include a cannula, wherein the cannula is flared shaped or funnel shaped. The treatment support system can further include an insertion tool wherein the insertion tool includes a plurality of sequentially sized slidable segments. The fluid delivery system can include a biocompatible material.

In an interrelated aspect, a treatment support system includes a fluid delivery system for directing a fluid to and from a treatment site, a portion of the fluid delivery system being configured to mechanically stabilize the treatment site while delivering fluid to and from the treatment site during articulation of the treatment site, the portion of the fluid delivery system being configured to expand from a first collapsed configuration to a second expanded stabilizing configuration during treatment and back to a collapsed configuration for removal from the treatment site.

In some variations, one or more features disclosed herein, including the following features, can optionally be included in any feasible combination. The portion of the fluid delivery system being configured to expand can include an open-cell sponge, a mesh, and/or a lattice. The mesh can include one or more flexible biocompatible materials including a polyester, a polypropylene, a polymer, a silk, a nylon and/or a nickel-titanium alloy. The mesh can include a helically wound braided tube. The mesh can include a sleeve shaped to conform to an anatomy of a portion of the treatment site. The treatment support system can further include a spacer configured to articulate with an inner surface of the treatment site. The expansion from the first collapsed configuration to the second expanded stabilizing configuration can occur when the fluid delivery system reaches a temperature at or near patient body temperature.

In an interrelated aspect, a treatment support system includes a fluid delivery system for directing a fluid to and from a treatment site including a portion of a joint; and an attachment feature attached to the fluid delivery system, the attachment feature being configured to prevent migration, kinking or clogging of the fluid delivery system.

In some variations, one or more features disclosed herein, including the following features, can optionally be included in any feasible combination. The fluid delivery system can further include a catheter or tubing. The treatment support system can further include a mesh including a sleeve shaped to conform and direct flow to or from an anatomy of a portion of the treatment site. The attachment feature can include a spacer configured to allow fluid flow and to protect retained implants during therapy. The spacer can be configured to mechanically stabilize the treatment site while delivering fluid to and from the treatment site during articulation of the treatment site.

### P Embodiments

P Embodiment 1. A treatment support system comprising: a fluid delivery system for directing a fluid to and from a treatment site, a portion of the fluid delivery system being configured to prevent migration of the fluid delivery system from the treatment site, the portion of the fluid delivery system being configured to collapse to a minimum cross-section for a minimally invasive transcutaneous deployment to the treatment site or minimally invasive transcutaneous removal from the treatment site.
P Embodiment 2. The treatment support system of P Embodiment 1, further comprising an inflatable structure configured to fill an opening of the treatment site during a treatment.
P Embodiment 3. The treatment support system of P Embodiment 2, wherein the inflatable structure comprises a balloon or a bladder, inflatable with gas and/or liquid.
P Embodiment 4. The treatment support system of P Embodiment 1, wherein the fluid delivery system comprises a catheter or tubing.
P Embodiment 5. The treatment support system of P Embodiment 4, wherein the catheter or tubing comprises an exterior attachment feature to removably attach the catheter or tubing to a portion of the treatment site during treatment.
P Embodiment 6. The treatment support system of P Embodiment 5, wherein the exterior attachment feature comprises a magnet, a suture, an adhesive, a resorbable material, a hook, and/or a ring.
P Embodiment 7. The treatment support system of P Embodiment 1, further comprising a cannula, wherein the cannula is flared shaped or funnel shaped.
P Embodiment 8. The treatment support system of P Embodiment 1, further comprising an insertion tool wherein the insertion tool comprises a plurality of sequentially sized slidable segments.
P Embodiment 9. The treatment support system of P Embodiment 1, wherein the fluid delivery system comprises a biocompatible material.
P Embodiment 10. A treatment support system comprising: a fluid delivery system for directing a fluid to and from a treatment site, a portion of the fluid delivery system being configured to mechanically stabilize the treatment site while delivering fluid to and from the treatment site during articulation of the treatment site, the portion of the fluid delivery system being configured to expand from a first collapsed configuration to a second expanded stabilizing configuration during treatment and back to a collapsed configuration for removal from the treatment site.
P Embodiment 11. The treatment support system of P Embodiment 10, wherein the portion of the fluid delivery system being configured to expand comprises an open cell sponge, a mesh, and/or a lattice.
P Embodiment 12. The treatment support system of P Embodiment 11, wherein the mesh comprises one or more flexible biocompatible materials comprising a polyester, a polypropylene, a polymer, a silk, a nylon and/or a nickel-titanium alloy.
P Embodiment 13. The treatment support system of P Embodiment 11, wherein the mesh comprises a helically wound braided tube.
P Embodiment 14. The treatment support system of P Embodiment 11, wherein the mesh comprises a sleeve shaped to conform to an anatomy of a portion of the treatment site.
P Embodiment 15. The treatment support system of P Embodiment 11, further comprising a spacer configured to articulate with an inner surface of the treatment site.
P Embodiment 16. The treatment support system of P Embodiment 15, wherein expansion from the first collapsed configuration to the second expanded stabilizing configuration occurs when the fluid delivery system reaches a temperature at or near patient body temperature.
P Embodiment 17. A treatment support system comprising: a fluid delivery system for directing a fluid to and from a treatment site comprising a portion of a joint; and an attachment feature attached to the fluid delivery system, the attachment feature being configured to prevent migration, kinking or clogging of the fluid delivery system.
P Embodiment 18. The treatment support system of P Embodiment 17, wherein the fluid delivery system further comprises a catheter or tubing.
P Embodiment 19. The treatment support system of P Embodiment 18, further comprising a mesh comprising a sleeve shaped to conform and direct flow to or from an anatomy of a portion of the treatment site.
P Embodiment 20. The treatment support system of P Embodiment 18, wherein the attachment feature comprises a spacer configured to allow fluid flow and to protect retained implants during therapy.
P Embodiment 21. The treatment support system of P Embodiment 20, wherein the spacer is configured to mechanically stabilize the treatment site while delivering fluid to and from the treatment site during articulation of the treatment site.

1.-65. (canceled)

66. A medical system for treating an infected wound space, the medical system comprising:
a cannula comprising:
a tubular portion comprising a lumen extending from a proximal opening in a proximal end region to a distal opening in a distal end region of the cannula; and
a distal flared region at the distal end region of the cannula, said distal flared region surrounding the distal opening and comprising an outer diameter greater than a diameter of the distal opening; and
a sponge, said sponge comprising:
a first portion sized and shaped for implantation within the wound space and located distally of the distal flared region of the cannula; and
a second portion sized and shaped to extend through each of the distal flared region, the distal opening, the lumen, and the proximal opening such that a proximal end portion of the sponge extends proximally beyond the proximal opening of the cannula,
wherein the sponge comprises a material having a tensile strength sufficient to prevent the sponge from tearing during retraction of the first portion in a proximal direction through the distal flared region into the distal opening and through at least a portion of the lumen of the cannula.

67. The system of claim 66, wherein the sponge is an open-cell foam.

68. The system of claim 66, wherein, upon implantation of the system in a patient, the distal flared region is positioned in the vicinity of the wound space, the first portion of the sponge is located within the wound space and the second portion of the sponge is located within the lumen of the cannula such that the proximal end portion of the sponge is accessible from outside the patient.

69. The system of claim 68, wherein the distal flange region is flexible and configured to be compressed to a reduced outer diameter during removal of said distal flange region from the patient through an opening in a skin of the patient after removal of the sponge from the wound space.

70. The system of claim 66, further comprising a proximal flange near the proximal end region of the cannula and having an outer diameter greater than an outer diameter of the tubular portion near the proximal end region of the cannula, said proximal flange adapted to remain outside the patient when the system is implanted and to resist the cannula being pushed into the patient.

71. The system of claim 66, further comprising a treatment delivery system for delivering fluid to the wound space while the medical system is implanted.

72. The system of claim 71, wherein the treatment delivery system provides instillation of fluid to, and/or aspiration using negative pressure of, the wound space.

73. The system of claim 71, wherein the treatment delivery system is manually or electronically controlled.

74. The system of claim 72, wherein the treatment delivery system comprises a source of negative pressure.

75. The system of claim 74, wherein the cannula and sponge provide fluid communication between the source of negative pressure and the wound space for fluid removal from the wound space.

76. The system of claim 72, wherein instillation of fluid to the wound space comprises delivery of a therapeutic fluid to the wound space.

77. The system of claim 76, wherein the therapeutic fluid comprises at least one of an antibiotic, an analgesic, an antifungal, or a combination thereof.

78. The system of claim 77, wherein said antibiotic comprises at least one of vancomycin or tobramycin.

79. The system of claim 66, wherein the wound space comprises one of a surgical site, a joint space, a traumatic bone injury, a fracture-related infection and osteomyelitis.

80. The system of claim 66, wherein the first portion of the sponge comprises a “Y” shape with two separate distal legs.

81. The system of claim 80, wherein each of the two separate distal legs of the “Y” shape is adapted to contact a condyle of a knee joint adjacent a patella.

82. A method of treating an infected wound space, the method comprising:
positioning a portion of a medical device within a patient adjacent the wound space, the medical device comprising a cannula and a sponge, such that after insertion, a proximal portion of the cannula resides outside the patient, a distal portion of the cannula is within the patient and an elongate body of the cannula extends through an opening in the patient,
wherein the cannula further comprises a lumen extending through the elongate body of the cannula from a proximal opening in the proximal portion of the cannula to a distal opening in the distal portion of the cannula, and wherein the distal portion comprises a distal flared region, said distal flared region surrounding the distal opening and comprising an outer diameter greater than a diameter of the distal opening;
positioning a first portion of the sponge within the wound space, wherein said first portion of the sponge is located distally of the distal flared region of the cannula while a second portion of the sponge extends proximally from the first portion through each of the distal flared region, the distal opening and the lumen towards the proximal opening of the cannula, wherein the sponge comprises a material having a tensile strength sufficient to prevent the sponge from tearing during retraction of the first portion in a proximal direction into the distal flared region, into the distal opening and through at least a portion of the lumen of the cannula;
applying reduced pressure to the wound space to withdraw a fluid from the patient;
removing the sponge from the patient by pulling on the proximal end portion to retract the first portion of the sponge proximally into the distal flared region, into the distal opening and through at least a portion of the lumen of the cannula; and
removing the distal flared portion of the cannula from the patient through the transdermal opening by compressing the distal flared portion to reduce the outer diameter as the distal flared portion is pulled through the opening.

83. The method of claim 82, further comprising instilling a therapeutic fluid into the wound space, and allowing the therapeutic agent to dwell within the wound space.

84. The method of claim 83, wherein said instilling the therapeutic agent, allowing the therapeutic agent to dwell, and applying reduced pressure constitutes a cycle of therapeutic treatment, and wherein said method comprises repeating a plurality of cycles of therapeutic treatment.

85. The method of claim 82, wherein said opening is a transdermal puncture.

86. The method of claim 82, wherein the cannula is inserted into the patent through the opening by passing the proximal portion of the cannula from inside the patient through the opening towards the outside of the patient.

87. The method of claim 82, wherein the cannula is percutaneously inserted from outside the patient through the opening.

88. The method of claim 82, wherein the therapeutic fluid comprises at least one of an antibiotic, an analgesic, an antifungal, or a combination thereof.

89. The method of claim 88, wherein said antibiotic comprises at least one of vancomycin or tobramycin.

90. The method of claim 82, wherein the wound space comprises one of an infected surgical site, an infected joint space, an infected traumatic bone injury, a fracture-related infection and osteomyelitis.

91. The method of claim 82, wherein the first portion of the sponge comprises a “Y” shape with two separate distal legs.

92. The system of claim 91, wherein said positioning the first portion of the sponge within the wound space comprises positioning each of the two separate distal legs of the “Y” shape to contact a condyle of a knee joint adjacent a patella.
